(19) United States
(12) Patent Application Publication
(10) Pub. No.: US 2006/0228326 A1
(43) Pub. Date: Oct. 12, 2006

(54) Active specific immunotherapy of cancer metastasis

(76) Inventors: Isaiah J. Fidler, Houston, TX (US); Zhongyun Dong, Sugar Land, TX (US); Weixin Lu, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/532,551

(22) PCT Filed: Oct. 21, 2003
(86) PCT No.: PCT/US03/33395

§ 371 Date: Mar. 30, 2006

Related U.S. Application Data
(60) Provisional application No. 60/420,209, filed on Oct. 22, 2002.
(60) Provisional application No. 60/453,330, filed on Mar. 10, 2003.

(51) Int. Cl. A61K 38/21; A61K 38/20; A61K 38/19; C12P 21/02; C12N 5/06

(52) U.S. Cl. 424/85.1; 424/85.2; 424/85.5; 424/85.6; 424/85.7; 435/69.5; 435/348

(57) ABSTRACT

The present invention provides for the treatment of a subject with occult brain metastasis. The treatment relies on administering to the subject a composition comprising an immunomodulatory polypeptide and a baculovirus-insect cell preparation. This composition has a unique ability to generate an anti-tumor immune response that is able to cross the blood-brain barrier.

The present application claims benefit of priority to U.S. Provisional Ser. No. 60/420,209, filed Oct. 22, 2002, and U.S. Provisional Ser. No. 60/453,330, filed Mar. 10, 2003, the entire contents of both being hereby incorporated by reference.

The government owns rights in the present invention pursuant to grant number the Cancer Center Support Core grant CA16672, Prostate Cancer grant CA90270, Ovarian Cancer grant CA93639, and Head and Neck Cancer grant CA37007 from the National Cancer Institute, National Institutes of Health, and grant RPG-98-332 (Z.D.) from the American Cancer Society.

### BACKGROUND OF THE INVENTION

A. Field of the Invention

The present invention relates generally to the fields of immunology and cancer biology. More particularly, it concerns the use of insect cell-immunomodulatory compositions to prevent or treat metastatic cancer in the brain.

B. Description of Related Art

In the United States, more than 170,000 patients develop brain metastasis annually (Posner, 1992; Loeffler et al., 1997). Despite recent advances in the diagnosis and treatment of brain metastases, the median survival of these patients is less than 1 year (Lewis, 1988; Zucker et al., 1978; Fidler et al., 1999). Clearly, new approaches for treating this fatal aspect of cancer are urgently needed.

Immunotherapy is an attractive and promising strategy for treatment of cancer (Rosenberg, 1997; Ostrand-Rosenberg et al., 1999). The goal of active, specific immunotherapy is to activate tumor-specific T cells and tumor-infiltrating macrophages (Ostrand-Rosenberg et al., 1999; Rosenberg, 2001) to destroy cancer cells in both primary tumors and metastatic lesions (Jaffee, 1999; Galea-Lauri et al., 1996). Although the central nervous system (CNS) has been considered to be an immunologically privileged site (Shirai, 1921; Murphy and Sturm, 1923; Grooms et al., 1977; Mitchell, 1989), recent studies indicate that tumors in the CNS can be partially or completely suppressed by active immunotherapy (Sampson et al., 1996; Fakhrai et al., 1996; Ashley et al., 1997; Okada et al., 1998; Visse et al., 1999).

The inventors have previously established a novel active immunotherapeutic system consisting of a recombinant baculovirus expression vector encoding IFN-β (H5BVIFN-β) (Kidd and Emery, 1993; Possee, 1997; Lu et al., 2002). In these studies, the inventors injected a preparation of lyophilized H5BVIFN-β into subcutaneous (s.c.) murine TV-2237M fibrosarcomas and K-1735M2 melanomas. A potent systemic immune response was induced, leading to immunologically-specific eradication of both injected primary tumors and uninjected lung metastases (Lu et al., 2002). However, the ability of this type of therapy to reach metastatic tumors in the brain has not been assessed.

### SUMMARY OF THE INVENTION

Thus, in accordance with the present invention, there is provided a method for preventing occult brain metastasis in a subject or treating a subject with occult brain metastasis comprising administering to said subject a composition comprising an immunomodulatory polypeptide and a baculovirus-insect cell preparation. The composition may be injected directly into a tumor or into tumor vasculature not located in the brain. The occult brain metastasis may be derived from a primary tumor in said subject's bone, liver, spleen, pancreas, lung, colon, testis, ovary, breast, cervix, prostate, and uterus. The method may further comprise a second or a third administration of said composition. The subject may be a human. The method may further comprise a second anti-cancer therapy, such as radiotherapy, chemotherapy, gene therapy or surgery. The subject may have previously received cancer therapy.

The composition may comprise between about 10⁵ and about 10⁷ insect cells. The composition may comprise intact or disrupted insect cells. The composition may be lyophilized and/or have been freeze/thawed. The immunomodulatory polypeptide may be expressed from a recombinant baculovirus vector in an insect cell. The immunomodulatory polypeptide may be IFN-α, IFN-β, IFN-γ, IL-1, IL-2, IL-6, IL-7, IL-12, IL-15, IL-16 or GM-CSF. The composition may also comprise an inflammatory stimulus. The inflammatory stimulus may be whole bacteria, endotoxin, or unmethylated DNA. The composition may comprise Spodoptera or Trichoplusia cells, or products of these cells resulting from disruption thereof. The composition may further comprise a tumor antigen, such as MAGE-1, MAGE-3, Melan-A, P198, P1A, gp100, TAG-72, p185^{HER2}, milk mucin core protein, carcinoembryonic antigen (CEA), P91A, p53, p21^{ras}, P210, BTA or tyrosinase. The tumor antigen may be expressed from a recombinant baculovirus vector in an insect cell.

In accordance with the present invention, there is also provided a method for preventing occult brain metastasis in a subject or treating a subject with occult brain metastasis comprising administering to said subject a composition comprising an immunomodulatory polypeptide and an inflammatory stimulus. The composition may be injected directly into a tumor or into tumor vasculature not located in the brain. The occult brain metastasis may be derived from a primary tumor in said subject's bone, liver, spleen, pancreas, lung, colon, testis, ovary, breast, cervix, prostate, and uterus. The method may further comprise a second or a third administration of said composition. The subject may be a human. The method may further comprise a second anti-cancer therapy, such as radiotherapy, chemotherapy, gene therapy or surgery. The subject may have previously received cancer therapy.

The composition may be lyophilized and/or have been freeze/thawed. The immunomodulatory polypeptide may be expressed from a recombinant baculovirus vector in an insect cell. The immunomodulatory polypeptide may be IFN-α, IFN-β, IFN-γ, IL-1, IL-2, IL-6, IL-7, IL-12, IL-15, IL-16 or GM-CSF. The inflammatory stimulus may be whole bacteria, endotoxin, or unmethylated DNA. The composition may comprise Spodoptera or Trichoplisia cells, or products of these cells resulting from disruption thereof. The composition may further comprise a tumor antigen, such as MAGE-1, MAGE-3, Melan-A, P198, P1A, gp100, TAG-72, p185^{HER2}, milk mucin core protein, carcinoembryonic antigen (CEA), P91A, p53, p21^{ras}, P210, BTA or tyrosinase. The tumor antigen may be expressed from a recombinant baculovirus vector in an insect cell.

There is also provided a method for preventing the development of occult brain metastasis in a subject comprising administering to the subject a composition comprising an immunomodulatory polypeptide and an inflammatory stimulus. Also provided is a method for preventing the development of occult brain metastasis in a subject comprising administering to the subject a composition comprising an immunomodulatory polypeptide and a baculovirus-insect cell preparation.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1. H5BVIFN-β therapy of brain metastasis. C3H/HeN mice were injected s.c. with either UV-2237M or K-1735M2 melanoma cells. One week later when the tumors reached the size of 4-5 mm in diameter, the mice were randomized into the following groups (n=10):control, tumors resected surgically, and tumors injected with 2 units of H5BVIFN-β preparation. The K-1735M tumors were injected a second time with H5BVIFN-β one week later. Six weeks after the complete regression (or resection) of the tumors, all mice were injected in the carotid artery with UV-2237M or K-1735M2 cells. The mice were killed when they became moribund. Surviving mice were killed on day 180. The brains were fixed, sectioned, and examined histologically. Note that H5BVIFN-β treatment of s.c. UV-2237M tumors prevented development of UV-2237M brain metastases but not K-1735M2 brain metastases. Conversely, H5BVIFN-β treatment of s.c. K-1735M2 tumors prevented development of K-1735M2 brain metastases but not UV-2237M brain metastases.

FIGS. 2A-E. Eradication of established s.c. tumors and occult brain metastases by H5BVIFN-β therapy. UV-2237M cells were injected s.c. into C3H/HeN mice. Five days later, the mice were randomized into two groups to receive intracarotid injections of either UV-2237M cells (FIGS. 2A-B) or K-1735M2 (FIGS. 2C-D). Two days later, each group was further randomized into 2 groups to receive injections of H5BVIFN-□ or PBS into the s.c. tumors. The size (diameter in mm) and incidence of s.c. tumors (the fraction adjacent to each line) are shown (FIGS. 2A and 2C). Moribund mice were killed and their brains were evaluated by histology for presence of metastases (FIG. 2E). Note that mice receiving H5BVIFN-β injection into UV-2237M s.c. tumor had no UV-2237M brain metastases but did have K-1735M2 metastases. Arrows indicate the time of intratumoral injection of H5BVIFN-β. *3 mice died before day 35.

FIGS. 3A-B. Eradication of s.c. tumors and brain metastases by H5BVIFN-β therapy is T cell dependent. C3H/HeN mice were injected s.c. with UV-2237M fibrosarcoma cells. When the tumors reached 3-5 mm in diameter (day 7), the mice were injected in the internal carotid artery with UV-2237M cells. Two days later, the mice were randomized to receive 3 i.p. injections on alternating days of 100 μl of PBS (control), PBS containing 200 μg isotype-matched rat IgG, anti-CD4, anti-CD8, or anti-CD4 plus anti-CD8 antibodies. One day after the first i.p. injection, s.c. UV-2237M tumors were injected intralesionally with 2 units of H5BVIFN-β cells. Control tumors were not injected but were resected once they reached 15 mm in diameter. All mice were killed when they became moribund. All surviving mice were killed on day 180. *P<0.001.

FIGS. 4A-B. Immunohistochemistry of brain metastases. C3H/HeN mice were injected s.c. with UV-2237M fibrosarcoma cells. On day 7 when the s.c. tumors reached 4-5 mm in diameter, the mice received intracarotid injections of UV-2237M cells. Two days later, the mice were randomized to receive 3 i.p. injections (on alternating days) of PBS (control). PBS containing 200 μg isotype-matched rat IgG, anti-CD4, anti-CD8, or anti-CD4 plus anti-CD8. One day after the first i.p. treatment (day 10), the s.c. tumors (in all treatment groups except control mice) were injected with 2 units of lyophilized H5BVIFN-β. Mice were killed on day 19 and the brains were processed for immunohistochemistry to identify the presence of CD4+ and/or CD8+ cells within brain metastases.

FIG. 5. Effect of IFN-β Insect Cell Preparations on Existing Lung Metastasis Following Resection of Primary Tumors. TV-2237m cells (2×10⁵/mouse) were s.c. injected into 20 C3H/HeN mice. On day 18 after tumor cell inoculation, the tumor-bearing mice were i.v. injected with 5×10⁴/mouse of UV-2237m cells. Five naïve mice were i.v. injected with UV-2237m cells as a control. One day later, the subcutaneous tumors were surgically resected, enzymatically dissociated, and irradiated (2,000 rads from the Cesium-137 source). On day 21, mice in which s.c. tumor were surgically removed were randomized into 4 groups and s.c. injected with PBS, 2×10⁶ lyophilized H5BVIFN-β, 5×10⁶ irradiated cells from UV-2237m tumors, or a mixture of H5BVIFN-β and 5×10⁶ irradiated cells. The treatment was repeated on day 28 and 35 after the subcutaneous tumor cell inoculation.

FIG. 6. Effect of IFN-13 Insect Cell Preparations on Exhisting Lung Metastsis. UV-2237m cells (5×10⁴/mouse) were injected into 40 C3H/HeN mice. On day 3 after the tumor cell inoculation, the mice were randomized into 4 groups and treated by s.c. injection of PBS, 2×10⁶ lyophilized H5BVIFN-β cells, 5×10⁶ irradiated UV-2237m cells (2000 rads from a Cesium-137 source), or H5BVIFN-β plus irradiated UV-2237m cells.

FIG. 7. Active Components of H5 Cells in IFN-β Therapy. UV-2237m cells (2×10⁵/mouse) were s.c. injected into C3H/HeN mice. On day 7 after tumor cell inoculation, the tumors were injected with PBS or 2×10⁶ lyophilized H5BVIFN-β, a mixture of 2×10⁴ units IFN-β and 2×10⁶ lyophilized H5 cells or components (lipid, protein, and/or DNA) extracted from 2×10⁶H5 cells. Subcutaneous tumors were measured once a week and the experiment was terminated on day 41 after tumor cell inoculation.

FIG. 8. Synergistic Effects of IFN-α and H5 Cells. UV-2237m cells (2×10⁵/mouse) were s.c. injected into C3H/HeN mice. On day 7 after tumor cell inoculation, the tumors were injected with PBS or 2×10⁶ lyophilized H5 cells, a mixture of 2×10⁶ lyophilized H5 cells and 1 or 2×10⁴ units of IFN-β or IFN-α. Subcutaneous tumors were measured once a week and the experiment was terminated on day 28 after tumor cell inoculation.

FIG. 9. Active Components of H5 Cells in IFN-α Therapy. UV-2237m cells (2×10⁵/mouse) were s.c. injected into 35 C3H/HeN mice. Seven days later, the tumors were injected with PBS, 2×10⁶ lyophilized H5BVIFN-β (positive control), a mixture of 2×10⁴ units of IFN-α and 2×10⁶lyophilized H5 cells, or cellular components (lipid, protein, and/or DNA) extracted from 2×10⁶H5 cells. Subcutaneous tumors were measured once a week and experiment was terminated on day 29 after tumor cell inoculation.

FIG. 10. Therapeutic Efficacy of H5 with IFN-α and -β. UV-2237m cells (2×10⁵/mouse) were s.c. injected into 30 C3H/HeN mice. On day 7 after tumor cell inoculation, the tumors were injected with PBS, 2×10⁴ units of IFN-α, 2×10⁴ units of IFN-γ, a mixture of 2×10⁶ lyophilized H5 cells and 2×10⁴ units of IFN-α, or a mixture of 2×10⁶ lyophilized H5 cells and 2×10⁴ units of IFN-γ. Subcutaneous tumors were measured once a week and data shown are up to day 28 after tumor cell inoculation.

FIGS. 11-12. Effect of H5 Cell IFN-α on Existing Lung Metastasis. C3H/HeN mice were s.c. and i.v. injected with 2×10⁵/mouse of UV2237m cells. On day 7 after the inoculation, s.c. tumors were resected. One day later, the mice were treated by s.c. injection of PBS, a mixture of 2×10⁶ lyophilized H5 cells and 2×10⁴ units of IFN-α, 10⁷ of irradiated UV-2237m cells prepared from subcutaneous tumors, or a mixture of 2×10⁶ lyophilized H5 cells, 2×10⁴ units of IFN-α, and 10⁷ of UV-2237m cells. The treatments were repeated once one week later. The experiment was terminated on day 20 after the therapy.

FIG. 13-14. H5 Cell Chronic Toxicity Study. Two experiments were performed to determine whether subcutaneous administration of H5BVIFN-β produces toxic effects on mice. In the first experiment, normal C3H/HeN mice were randomized into 4 groups (10 mice/group) and injected s.c. with PBS or lyophilized H5BVIFN-β (2×10⁶, 20×10⁶, or 40×10⁶ cells/injection) for 2 times 1 week apart. Body weight of each mouse was measured once for 6 weeks (FIG. 13). After 6 weeks, three mice per group were euthanized and lungs, liver, kidneys, spleen, heart, brain, and a fragment of small intestine were collected for each mouse for histologic study. In the second experiment, potential toxic effects of long-term administration of H5BVIFN-β were determined. C3H mice were randomized into 3 groups (10 mice/group) and injected s.c. with PBS or with lyophilized preparation of 20×10⁶ H5BVIFN-β in 100 μl PBS/mouse once a week for 6 weeks or 12 weeks. Body weight of each mouse was measured once a week (FIG. 14). After 6 weeks or 12 weeks, three mice per group were euthanized and lungs, liver, kidneys, spleen, heart, brain, and a fragment of small intestine were collected for each mouse for histologic study.

FIG. 15. H5 Cell Acute Toxicity Study. C3H/HeN female mice at 12 weeks of age were divided into six groups: Groups 1-3 were tumor-bearing mice (5 mice per group), and Groups 4-6 were normal mice (5 mice per group). Tumor-bearing mice were injected with UV-2237m cells s.c. For each mouse, 4 sites were injected. When each tumor reached approximately 1 cm in diameter, mice were injected with materials detailed in the treatment section. Treatment was as follows: Groups 1 and 4 were treated 1 ml of PBS; Groups 2 and 5 were treated with 1 ml of PBS with 10⁷ lyophilized H5 cells plus 2×10⁴ units of murine IFN-α; Groups 3 and 6 were treated with 1 ml of PBS with 5×10⁷ lyophilized H5 cells plus 2×10⁴ units of murine IFN-α.

### DESCRIPTION OF ILLUSTRATIVE EMBODIMENTS

In previous studies, the present inventors reported that insect cell preparations possess adjuvant properties. In addition, the combination of insect cell compositions with specific immunomodulators resulted in a synergistic anti-cancer effect. Two alternate embodiments were described. The first involves the use of insect cells or insect cell compositions, alone or in conjunction with immunomodulators, antigens or antigenic preparations that were added to the cell compositions. The second embodiment relies on the expression of the immunomodulator or antigen within the insect cells using a baculovirus vector. In both contexts, the combination of immune stimulatory molecules with the insect cell compositions provided surprising results. The present invention extends this earlier work by applying the insect cell compositions to the treatment of brain metastasis.

Traditionally, the brain has been considered to be an immune privileged site (Shirai, 1921; Murphy and Sturm, 1923; Grooms et al., 1977; Mitchell, 1989); however, several recent studies dealing with brain tumors suggest that the blood-brain barrier is not an absolute barrier for lymphocytes and macrophages (Sampson et al., 1996; Okada et al., 1998). In fact, activated T cells in the systemic circulation have been shown to freely traverse the barrier (Wekerle et al., 1987). Further, subcutaneous injection with IFN-γ, interleukin-7 (IL-7), or B7-1-gene-transfected rat glioma cells has been shown to lead to the regression of occult intracerebral glioma isografts (Visse et al., 1999). Similarly, subcutaneous immunization with granulocyte-macrophage colony-stimulating factor (GM-CSF)-gene-engineered tumor cells have been shown to induce immune responses that protect mice from a second challenge by tumor cells implanted in the periphery and the brain (Sampson et al., 1996). Of interest was the inventors' previous finding that, like the GM-CSF study, insect cells engineered to express IFN-β showed an ability to induce immunologic memory that was specific for a particular cancer cell type.

The present inventors sought to determine whether the IFN-β/insect cell composition could have an effect on occult brain metastasis. Following intralesional injection of a lyophilized preparation of H5 insect cells, regression of subcutaneous tumors was initiated by active-specific T cells (CD4+, CD8+) that crossed the blood-brain barrier and infiltrated and destroyed the metastases. Systemic administration of antibodies against CD4 and/or CD8 antigens abrogated the active-specific therapeutic effects of in both s.c. tumors (Lu et al., 2002) and brain metastases (this study). These data are consistent with those from studies on the regression of lung metastases (Lu et al., 2002) and suggest that the subsets of T cells required to eradicate tumors in the brain may vary with the cytokine used to initiate the therapy (Sampson et al., 1996) and the type of tumor growing in the brain.

A therapy using lyophilized preparation of H5BVIFN-β, but not its individual component (H5 cells or IFN-β), was necessary for inducing the immune protection against the intracranial challenge. This is based on the observation that the induction of the immune protection depends on the elimination of s.c. tumors, and only treatment with H5BVIFN-β, but not H5 cells nor IFN-β, can eradicate s.c. tumors (22). However, the exact components in the preparation of H5BVIFN-β that augmented the immune stimulatory effects of IFN-β remain unknown. Recent studies demonstrate that the innate immune response against pathogens is dependent upon pattern recognition receptors on antigen-presenting cells (30-33). These receptors recognize common patterns shared by bacteria or viruses that are not present on normal host cells. The triggering of pattern recognition receptors can lead to expression of high levels of costimulatory molecules, such as CD80 and CD86, that prime and activate antigen-specific T cells, and to the secretion of proinflammatory cytokines, e.g., IL-1, IL,6, IL-12, tumor necrosis factor-alpha (TNF-α), GM-CSF, and type I IFN (30-33).

Several recent studies show that the unmethylated CpG motifs in the insect cell DNA, by inducing type I interferon production, can augment T cell responses to specific antigens (34-36). However, in the present study, the intratumoral injection of H5 cells, or in other studies, H5 cells transduced with a baculoviral vector expressing GM-CSF (data not shown), had minimal therapeutic effects on UV-2237M tumors. These data suggest that other components in the H5 cells serve as an adjuvant to augment the specific immune response against tumor cells. The present data do not exclude the possibility that other inflammatory stimuli, such as whole bacteria, endotoxins, and unmethylated DNA, combined with IFN-β could be as effective as insect cells in eradicating tumors. Furthermore, in the present study, only the role of insect cells plus IFN-β in eradicating tumors was investigated. Since IFN-β and IFN-α share type I IFN receptors, it is possible that IFN-α could substitute for IFN-β.

In summary, the inventors have shown that the injection of an insect cell/IFN-β into established s.c. tumors can eradicate the both the primary skin tumors and related occult brain metastases. Unlike previous studies using genetically modified tumor cells, the success of this therapy does not require the transfection of tumor cells or the use of tumor antigens. The eradication of the brain metastases by insect cell/IFN-β therapy was not associated with any detectable behavioral changes in the treated tumor-bearing mice. Even 10 consecutive weekly s.c. injections of 20 units of H5BVIFN-β did not lead to demonstrable toxicity. Thus, this constitutes a surprising extension of the utility of the earlier work with this composition.

### A. ANTI-TUMOR VACCINATION

Neoplastic or tumor cells generally express altered protein on their surface in the context of MHC Class I that may be detected by the immune system as foreign thus leading to the induction of an immune response. Frequently, the difficulty in inducing an anti-tumor response is not in establishing that a tumor antigen is present and detectable by immune surveillance. Rather, the problem centers on recruiting the necessary cells to the area and providing the cells with the proper secondary signals necessary for the development of an effective immune response. The adjuvant properties of the instant invention initiate the recruitment of immune cells into the tumor and provide for the recognition of tumor antigens generally leading to the ultimate regression of the tumor. A further benefit is that tumor infiltration by lymphocytes facilitates the creation of memory cells. Thus, if tumor cells have metastasized or if the tumor recurs, a subpopulation of lymphocytes can readily be dispatched to deal with subsequent challenges or metastatic cells. In particular, the present invention addresses the situation where the metastatic cells are located in the brain.

An added benefit of the disclosed system is that the preparation may be engineered to comprise recombinant proteins in the insect cell composition. Therefore, in a particular embodiment of the invention, the insect cell preparation is transformed with a expression vector, i.e., baculovirus comprising the gene for human IFN-β. A preparation of these cells may be directly introduced into the tumor, thus leading not only to the recruitment and activation of the immune cells by the adjuvant, but, in addition, the further benefit accorded by the inclusion of an secondary agent in the preparation. Other immunogenic molecules, such as tumor antigens, may be included in the insect cell composition.

It is contemplated that antitumor vaccination may occur by a variety of routes. In one embodiment of the instant invention, an insect cell composition is injected directly into a tumor in order to induce the recruitment of immune cells. It is envisioned that the formulation may comprise untransformed cells that are mixed with immunomodulatory proteins capable of enhancing immune cell recruitment, activation or proliferation, or that the insect cells may also contain exogenous DNA and thus be capable of expressing the immunomodulators. Though initially thought to be of limited value against metastatic disease, this approach has now been shown to induce a systemic response against remote (e.g., metastatic) cancer, even on the other side of the blood-brain barrier.

Related U.S. Pat. No. 6,342,216 and U.S. Ser. No. 09/872,162 are both hereby incorporated by reference in their entirety.

### B. INSECT CELLS

The term “insect cells” means insect cells from the insect species which exhibit adjuvant properties when introduced into a host organism or when contacted by immune cells. In certain embodiments of the instant invention, it is contemplated that insect cells comprise cells which are subject to baculovirus infection. For example: Autographa californica, Bombyx mori, Spodoptera frugiperda, Choristoneura funiferana, Heliothis virescenis, Heliothis zea, Orgyia pseudotsugata, Lymantira dispar, Plutelia xylostella, Malacostoma disstria, Trichoplusia ni, Pieris rapae, Mamestra configurata and Hyalophora cecropia. See U.S. Pat. No. 5,498,540 and 5,759,809, incorporated herein by reference. In a particular embodiment, the insect cells are H5 insect cells (Invitrogen, Sorrento, Calif.), derived from Trichoplusia ni. Such insect cells may be used in an intact form, or may be used following lyophilization or freeze-thaw cycles.

It is envisioned that a number species of insects possess cells or cell extracts that when introduced into a mammalian host would exhibit classic adjuvant properties. It is further contemplated that it is well within the capabilities of a person of ordinary skill in the art to screen alternate species, not expressly disclosed herein, for such properties.

Insect cells may be cultured according to standard techniques, such as in IPL-41 medium (JRH Biosciences, Inc.) with or without 10% fetal calf serum (Hyclone Laboratories, Inc.) as described in U.S. Pat. No. 5,759,809. A exemplary procedure for suspension cell cultures of H5 cell is, in brief, as follows. Adherent H5 cells are transferred from tissue culture flasks into spinner flasks. Serum free medium (Excell 400 medium from JRH BioSciences) supplemented with heparin is used to reduce cell aggregation. The cells are grown for several passages until they are >95% viable and have a doubling time between 18 and 24 hr. At this point, the cells are weaned from heparin. If the cells continue to grow in suspension without the addition of heparin they may be indefinitely maintained as a suspension until transformation. An alternative procedure for culturing insect cells in media containing fish serum has recently been described. See U.S. Pat. No. 5,498,540, incorporated herein by reference. For embodiments requiring transformed cells, cultured insect cells may be transfected with recombinant baculovirus or other expression vectors-by standard protocols. See, e.g., U.S. Pat. No. 5,759,809, incorporated herein by reference.

### C. BACULOVIRUS EXPRESSION VECTORS

Because of the relative simplicity of technology, capacity for large inserts, high expression levels of biologically functional recombinant protein, and ease of purification, the baculovirus expression vector system (BEVS) is one of the most powerful and versatile eukaryotic expression systems available. Compared to other higher eukaryotic expression systems, the most distinguishing feature of BEVS is its potential to achieve high levels of expression of a cloned gene. Consequently, in situ inoculation of tumors with insect cells infected with recombinant baculovirus encoding immunomodulating cytokine genes, antigens or should provide high local concentrations of cytokines to kill tumor cells and to elicit immune response, and should also enhance immunity per se since insect cells are heterologous to mammalian hosts.

1. Infection with Baculoviral Vectors

In certain embodiments of the invention, the nucleic acid encoding a selected non-surface expressed protein or peptide may be integrated into a baculovirus expression vector. Such vectors are useful tools for the production of proteins for a variety of applications (Summers and Smith, 1987; O'Reilly et al., 1992; also U.S. Pat. No. 4,745,051 (Smith and Summers), U.S. Pat. No. 4,879,236 (Smith and Summers), U.S. Pat. No. 5,077,214 (Guarino and Jarvis), U.S. Pat. No. 5,155,037 (Summers), U.S. Pat. No. 5,162,222, (Guarino and Jarvis), U.S. Pat. No. 5,169,784 (Summers and Oker-Blom) and U.S. Pat. No. 5,278,050 (Summers), each incorporated herein by reference). Baculovirus expression vectors are recombinant insect vectors in which the coding region of a particular gene of interest is placed behind a promoter in place of a nonessential baculoviral gene. The classic approach used to isolate a recombinant baculoviuus expression vector is to construct a plasmid in which the foreign gene of interest is positioned downstream of the polyhedriiz promoter. Then, via homologous recombination, that plasmid can be used to transfer the new gene into the viral genome in place of the wild-type polyhedrin gene (Summers and Smith, 1987; O'Reilly et al., 1992).

The resulting recombinant virus can infect cultured insect cells and express the foreign gene under the control of the polyhedrin promoter, which is strong and provides very high levels of transcription during the very late phase of infection. The strength of the polyhedrin promoter is an advantage of the use of recombinant baculoviruses as expression vectors because it usually leads to the synthesis of large amounts of the foreign gene product during infection.

Autographa californica multinucleocapsid nuclear polyhedrosis virus (AcMNPV) is unusual among baculoviruses because it displays a wider host range than most baculoviruses (Martignoni et al., 1982). AcMNPV is the most extensively studied baculovirus and its genome sequence is known (Ayres et al., 1994). It is distinguished by a unique biphasic life cycle in its lepidopteran host insect (reviewed in Blissard and Rohrmann, 1990). Infection produces high titers of two forms of progeny virus, budded virus (BV) and occlusion derived virus (ODV).

Two routes, adsorptive endocytosis (or viropexis) and direct fusion of BV envelope with plasma membrane, are proposed for entry of BV into cultured cells. Although BV may enter cells by fusion (Volkman et al., 1986), the majority of data indicates that the primary route is by adsorptive endocytosis (Charlton and Volkman, 1993).

2. Expression of Cloned Genes from Baculovirus Promoters and Enhancers

In certain aspects of the present invention, baculovirus vectors which are designed for the expression of a desired gene or genes are required. Thus, particular embodiments may require a selected nucleic acid segment to be operably linked to control sequences, such as promoters and enhancers. In the context of positioning nucleic acid segments and sequence regions in combination, the term “operably linked” will be understood to mean connected so as to form a single, contiguous nucleic acid sequence, wherein the promoters, enhancers and other control sequences are positioned and oriented in a manner to provide optimal expression of the gene. It will be understood that promoters are DNA elements which when positioned functionally upstream of a gene leads to the expression of that gene. Each heterologous gene in the vector of the present invention is functionally positioned downstream of a promoter element.

In transient systems, the gene of interest is introduced into the cell by infection with a recombinant virus, for example baculovirus. In the most widely used baculovirus systems, the gene of interest is under the control of the polyhedrin promoter. The polyhedrin promoter is a very late promoter, which means that the expression of the gene of interest does not start until the late phase of the baculovirus infection. The expression levels are high, but transient as the baculovirus infection eventually leads to cell death.

3. Baculoviral Promoters and Enhancers

There are four distinct phases of a baculovirus infection, termed immediate-early, delayed-early, late and very late. Therefore, different baculovirus genes may be classified according to the phase of the viral infection during which they are expressed. Also there are a class of genes which have been defined as early genes, which have not been subcatagorized as either immediate-early or delayed-early. Different classes of promoters control each class of gene.

Immediate early promoters are distinguished by needing only host cell factors to drive expression. Examples are the ie1 (Guarino and Summers, 1987), ieN ie2 (Carson et al., 1991) and ie0 promoters. Delayed early promoters are distinguished by needing only products of the immediate-early genes, in addition to host cell factors to drive expression. Examples are the 39K (Guarino and Smith, 1991) and gp64 (Blissard and Rohrmann, 1989; Whitford et al., 1989) promoters. Early promoters have not been placed into the specific immediate-early of delayed-early class. Examples include the DA26, ETL and 35K promoters.

Late promoters requires products of the delayed-early and immediate-early genes, as well as other host cell factors, to drive expression. Examples are the gp64 (Blissard and Rohrmann, 1989; Whitford et al., 1989) and capsid (p39; Thiem and Miller, 1989) promoters. Very late promoters requires a number of baculovirus gene products, in addition to other host cell factors, to drive expression. Examples of promoters from this class are the polyhedrin (Hooft van Iddekinge et al., 1983) and the p10 (Kuzio et al. 1984) promoters. The best characterized and most often used baculoviral promoter is the polyhedrin promoter. The use of the polyhedrin promoter is a preferred embodiment of the present invention.

Enhancers are DNA elements which can be positionally located to enhance transcription from a given promoter. Enhancers which are active in insect cells to drive transcription are preferred in the present invention. Preferred are viral enhancers, and most preferred are baculoviral enhancers. Examples of baculoviral enhancers include hr1, hr2, hr3, hr4 and hr5 (Guarino et al., 1986).

4. Marker Genes and Screening

In certain aspects of the present invention, specific cells may be tagged with specific genetic markers to provide information about the infected, transduced or transformed cells. Therefore, the present invention also provides recombinant candidate screening and selection methods which are based upon whole cell assays and which, preferably, employ a reporter gene that confers on its recombinant hosts a readily detectable phenotype that emerges only under conditions where a general DNA promoter positioned upstream of the reporter gene is functional. Generally, reporter genes encode a polypeptide (marker protein) not otherwise produced by the host cell which is detectable by analysis of the cell culture, e.g. by fluorometric, radioisotopic or spectrophotometric analysis of the cell culture.

In other aspects of the present invention, a genetic marker is provided which is detectable by standard genetic analysis techniques, such as DNA amplification by PCR™ or hybridization using fluorometric, radioisotopic or spectrophotometric probes.

Exemplary marker genes encode enzymes such as esterases, phosphatases, proteases (tissue plasminogen activator or urokinase) and other enzymes capable of being detected by their activity, as will be known to those skilled in the art. Contemplated for use in the present invention is green fluorescent protein (GFP) as a marker for transgene expression (Chalfie et al., 1994). The use of GFP does not need exogenously added substrates, only irradiation by near UV or blue light, and thus has significant potential for use in monitoring gene expression in living cells.

Other examples are chloramphenicol acetyltransferase (CAT) which may be employed with a radiolabeled substrate, firefly and bacterial luciferase, and the bacterial enzymes β-galactosidase and β-glucuronidase. Other marker genes within this class are well known to those of skill in the art, and are suitable for use in the present invention.

Another class of marker genes which confer detectable characteristics on a host cell are those which encode polypeptides, generally enzymes, which render their transformants resistant against toxins. Examples of this class of marker genes are the neo gene (Colberre-Garapin et al., 1981) which protects against toxic levels of the antibiotic G418, the gene conferring streptomycin resistance (U.S. Pat. No. 4,430,434), the gene conferring hygromycin B resistance (Santerre et al., 1984; U.S. Pat. Nos. 4,727,028, 4,960,704 and 4,559,302), a gene encoding dihydrofolate reductase, which confers resistance to methotrexate (Alt et al., 1978) and the enzyme HPRT, along with many others well known in the art (Kaufman, 1990).

### D. INFLAMMATORY STIMULI

1. Whole Bacteria and Endotoxins

Endotoxins are part of the outer membrane of the cell wall of Gram-negative bacteria Endotoxins are invariably associated with Gram-negative bacteria whether the organisms are pathogens or not. Although the term “endotoxin” is occasionally used to refer to any cell-associated bacterial toxin, it is properly reserved to refer to the lipopolysaccharide complex associated with the outer membrane of Gram-negative bacteria such as E. coli, Salmonella, Shigella, Pseudomonas, Neisseria, Haemophilus, and other leading pathogens.

The biological activity of endotoxins is associated with the lipopolysaccharide (LPS). Toxicity is associated with the lipid component (Lipid A) and immunogenicity is associated with the polysaccharide components. The cell wall antigens (O antigens) of Gram-negative bacteria are components of LPS. LPS elicits a variety of inflammatory responses in an animal. Because it activates complement by the alternative (properdin) pathway, it is often part of the pathology of Gram-negative bacterial infections.

In vivo, Gram-negative bacteria probably release minute amounts of endotoxin while growing. It is known, that small amounts of endotoxin may be released in a soluble form, especially by young cultures. However, for the most part, endotoxins remain associated with the cell wall until disintegration of the bacteria. In vivo, this results from autolysis of the bacteria, external lysis mediated by complement and lysozyme, and phagocytic digestion of bacterial cells.

Compared to the classic exotoxins of bacteria, endotoxins are less potent and less specific in their action, since they do not act enzymatically. Endotoxins are heat stable (boiling for 30 minutes does not destabilize endotoxin), but certain powerful oxidizing agents such as superoxide, peroxide and hypochlorite, degrade them. Endotoxins, although antigenic, cannot be converted to toxoids.

2. Unmethylated DNA

Bacterial DNA has been reported to stimulate mammalian immune responses (e.g., Krieg et al., 1995). One of the major differences between bacterial DNA, which has potent immunostimulator effects, and vertebrate DNA, which does not, is that bacterial DNA contains a higher frequency of unmethylated CpG dinucleotides than does vertebrate DNA. Select synthetic oligodeoxynucleotides (ODN) containing unmethylated CpG motifs (CpG ODN) have been shown to have an immunologic effects and can induce activation of B cells, NK cells and antigen-presenting cells (APCs) such as monocytes and macrophages (Krieg, A. M., et al., 1995). It can also enhance production of cytokines known to participate in the development of an active immune response, including tumor necrosis factor-.alpha., IL-12 and IL-6 (e.g., Klinman D. M., et al., 1996).

CpG DNA induces proliferation of almost all (>95%) B cells and increases immunoglobulin (Ig) secretion. This B cell activation by CpG DNA is T cell independent and antigen non-specific. However, B cell activation by low concentrations of CpG DNA has strong synergy with signals delivered through the B cell antigen receptor for both B cell proliferation and Ig secretion (Krieg et al., 1995). This strong synergy between the B cell signaling pathways triggered through the B cell antigen receptor and by CpG DNA promotes antigen specific immune responses. In addition to its direct effects on B cells, CpG DNA also directly activates monocytes, macrophages, and dendritic cells to secrete a variety of cytokines, including high levels of IL-12 (Klinman et al., 1996; Halpern et al., 1996; Cowdery et al., 1996). These cytokines stimulate natural killer (NK) cells to secrete gamma-interferon (IFN-γ) and have increased lytic activity (Klinman et al., 1996; Cowdery et al., 1996; Yamamoto et al, 1992; Ballas et al., 1996). Overall, CpG DNA induces a Th1 like pattern of cytokine production dominated by IL-12 and IFN-γ. with little secretion of Th2 cytokines (Klinman et al., 1996).

The binding of DNA to cells has been shown to be similar to a ligand receptor interaction: binding is saturable, competitive, and leads to DNA endocytosis and degradation into oligonucleotides (Benne, R. M., et al., 1995). Like DNA, oligodeoxyribonucleotides are able to enter cells in a process which is sequence, temperature, and energy independent (Jaroszewski and Cohen, 1991). Lymphocyte oligodeoxyribonucleotide uptake has been shown to be regulated by cell activation (Krieg et al., 1991).

The cytokines that are induced by unmethylated CpG oligonucleotides are predominantly of a class called “Th1” which is most marked by a cellular immune response and is associated with IL-12 and IFN-γ and production of IgG2a antibody. The other major type of immune response is termed as Th2 immune response, which is associated with more of an IgG1 antibody immune response and with the production of IL4, IL-5 and IL-10. In general, it appears that allergic diseases are mediated by Th2 type immune responses and autoimmune diseases by Th1 immune response. Based on the ability of the combination of CpG oligonucleotides and immunopotentiating cytokine to shift the immune response in a subject from a Th2 (which is associated with production of IgE antibodies and allergy and is produced in response to GM-CSF alone) to a Th1 response (which is protective against allergic reactions), an effective dose of a CpG oligonucleotide and immunopotentiating cytokine can be administered to a subject to treat or prevent an allergy.

Bacterial DNA, but not vertebrate DNA, has direct immunostimulatory effects on peripheral blood mononuclear cells (PBMC) in vitro (Messina et al., 1991; Tokanuga et al., 1994). These effects include proliferation of almost all (>95%) B cells and increased inmmunoglobulin (Ig) secretion (Krieg et al., 1995). In addition to its direct effects on B cells, CpG DNA also directly activates monocytes, macrophages, and dendritic cells to secrete predominantly Th 1 cytokines, including high levels of IL-12 (Klinman et al., 1996; Halpern et al., 1996; Cowdery et al., 1996). These cytokines stimulate natural killer (NK) cells to secrete γ-interferon (IFN-γ) and to have increased lytic activity (Klinman et al., 1996; Cowdery et al., 1996; Yamamoto et al., 1992; Ballas et al., 1996) These stimulatory effects have been found to be due to the presence of unmethylated CpG dinucleotides in a particular sequence context (CpG-S motifs) (Krieg et al., 1995). Activation may also be triggered by addition of synthetic oligodeoxynucleotides (ODN) that contain CpG-S motifs (Tokunaga et al., 1988; Yi et al., 1996; Davis et al., 1998).

### E. IMMUNE RESPONSE

The primary role of the subject matter of the instant invention is in the induction of an effective protective immune response, in particular, one that can cross the blood-brain barrier. A significant component of the claimed compositions is the ability of the composition to preferentially activate and induce the proliferation and/or recruitment of immune cells. The adjuvant properties of an insect cell or insect cell extract composition including a cytokine facilitate just such an immunologic response. In addition, it is envisioned that the compositions of the instant invention may further comprise antigenic components. The combination of an insect cell or insect cell extract composition and an immunomodulator, optionally further including an antigenic agent, facilitate the establishment of the desired immunological response and allow for the creation of immunologic memory.

1. Antigens

In one aspect, the invention provides a molecule or compound comprising an antigenic or immunogenic epitope. Compounds or molecules comprising an immunogenic epitope are those agents capable of inducing an immune response. An “immunogenic epitope” is defined as a part of an agent that elicits an immune response when the whole agent is the immunogen. These immunogenic epitopes are generally confined to a few loci on the molecule. For the purposes of the instant invention, the term “immunogen” or “immunogenic epitope” is not confined to the induction of solely a humoral or solely a cellular response. Rather, the term is used to denote the capability of a compound, molecule or agent to induce either or both a cellular and a humoral immune response.

As to the selection of molecules, compounds or agents bearing an immunogenic epitope it is well known in that art that specific conformations preferentially lead to the induction of a specific form of immune response. For example, peptides capable of eliciting protein-reactive sera as frequently represented in the primary sequence of a protein, can be characterized by a set of simple chemical rules, and are confined neither to immunodominant regions of intact proteins (i.e., immunogenic epitopes) nor to the amino or carboxyl terminals. For instance, 18 of 20 peptides designed according to these guidelines, containing 8-39 residues covering 75% of the sequence of the influenza virus hemagglutinin HA1 polypeptide chain, induced antibodies that reacted with the HA1 protein or intact virus; and 12/12 peptides from the MuLV polymerase and 18/18 from the rabies glycoprotein induced antibodies that precipitated the respective proteins.

U.S. Pat. No. 4,554,101, (Hopp) incorporated herein by reference, teaches the identification and/or preparation of epitopes from primary amino acid sequences on the basis of hydrophilicity. Through the methods disclosed in Hopp, one of skill in the art would be able to identify epitopes from within an amino acid sequence.

Numerous scientific publications have also been devoted to the prediction of secondary structure, and/or to the identification of epitopes, from analyses of amino acid sequences (Chou and Fasman, 1974a,b; 1978a,b, 1979). Any of these may be used, if desired, to supplement the teachings of Hopp in U.S. Pat. No. 4,554,101.

Moreover, computer programs are currently available to assist with predicting immunogenic portions and/or epitopic core regions of proteins. Examples include those programs based upon the Jameson-Wolf analysis (Jameson and Wolf, 1988; Wolf et al., 1988), the program PepPlot® (Brutlag et al, 1990; Weinberger et al., 1985), and/or other new programs for protein tertiary structure prediction (Fetrow and Bryant, 1993). Another commercially available software program capable of carrying out such analyses is MacVector (IBI, New Haven, Conn.).

Because of the protein expressing capabilities of the insect cells of the instant invention, it will often be desirable to provide a composition in which the insect cells also encompass an protein expressed in the context of an expression vector. In such an embodiment, immunogenic epitope-bearing peptides and polypeptides of the invention designed according to the above guidelines preferably contain a sequence of at least seven, more preferably at least nine and most preferably between about 15 to about 30 amino acids. However, peptides or polypeptides comprising a larger portion of an amino acid sequence of a polypeptide of the invention, containing about 30 to about 50 amino acids, or any length up to and including the entire amino acid sequence of the functional protein also are considered epitope-bearing peptides or polypeptides of the invention and also are useful for inducing the desired immune response. Preferably, the amino acid sequence of the epitope-bearing peptide is selected to provide substantial solubility in aqueous solvents (i.e., the sequence includes relatively hydrophilic residues and highly hydrophobic sequences are preferably avoided); and sequences containing proline residues are particularly preferred.

While in preferred embodiments of the invention, proteins are expressed by the transformed cells within the insect cell composition, it is also contemplated that native proteins or peptides or proteins produced by other means may be combined with the insect cell composition. The epitope-bearing peptides and polypeptides of the invention may thus be produced by any conventional means for making peptides or polypeptides including recombinant. For instance, a short epitope-bearing amino acid sequence may be fused to a larger polypeptide which acts as a carrier during recombinant production and purification. Epitope-bearing peptides also may be synthesized using known methods of chemical synthesis. For instance, Houghten et al. (1985) has described a simple method for synthesis of large numbers of peptides, such as 10-20 mg of 248 different 13 residue peptides representing single amino acid variants of a segment of the HA1 polypeptide which were prepared and characterized (by ELISA-type binding studies) in less than four weeks. This “Simultaneous Multiple Peptide Synthesis (SMPS)” process is further described in U.S. Pat. No. 4,631,211 to Houghten et al. (1986). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously. (Houghten et al, 1986).

Immunogenic epitope-bearing peptides are identified according to methods known in the art. For instance, Geysen et al. (1984) discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. (1984) with a resolution of seven amino acids by synthesis of an overlapping set of all 208 possible bexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 and Geysen (1987) further describes this method of identifying a peptide bearing an immunogenic epitope of a desired protein.

The immunogen or antigenic agent of the instant invention is contemplated to be or be derived from an agent or pathogen that causes some form of damage, injury, harm, morbidity or mortality to the host. As a result, an immunogen need not be an external agent but may be either a transformed or neoplastic cell. Further, the immunogen or antigenic agent need not be a living pathogen. Therefore, while an immunogen or agent would clearly constitute a bacteria, rickettsial, fungi, algae, protozoan, metazoan, helminth, other pathogenic organism or derivative thereof, it is also envisioned that the term would encompass any toxin, poison, virus, virion, virioid, prion or compound capable of doing harm to the host or to which it would be desirable to direct an immune response against.

The instant invention provides an adjuvant formulation that the skilled artisan will recognize as applicable to any number of cancers. The adjuvant composition may be provided in a formulation in which tumor antigens are either admixed with the insect cells or insect cell compositions or wherein the tumor antigen is expressed by the insect cells to be administered. An example of tumor antigens specifically contemplated for use in the context of the instant invention include MAGE-1, MAGE-3, Melan-A, P198, P1A, gp100, TAG-72, p185^{HER2}, milk mucin core protein, carcinoembryonic antigen (CEA), P91A, p53, p21^{ras}, P210, BTA and tyrosinase. Table 1 sets forth a more extensive, exemplary list of tumor antigens that may be employed in the context of the invention.

TABLE 1
Marker Antigens of Solid Tumors

| Tumor Site | Antigen Identity/Characteristics |
|---|---|
| A: Gynecological | |
| GY | ‘CA 125’ >200 kD mucin GP |
| ovarian | 80 Kd GP |
| ovarian | ‘SGA’ 360 Kd GP |
| ovarian | High Mᵣ mucin |
| ovarian | High Mᵣ mucin/glycolipid |
| ovarian | NS |
| ovarian | NS |
| ovarian | High Mᵣ mucin |
| ovarian | High Mᵣ mucin |
| GY | 7700 Kd GP |
| ovarian | ‘gp 68’ 48 Kd GP |
| GY | 40, 42 kD GP |
| GY | ‘TAG-72’ High Mᵣ mucin |
| ovarian | 300-400 Kd GP |
| ovarian | 60 Kd GP |
| GY | 105 Kd GP |
| ovarian | 38-40 kd GP |
| GY | ‘CEA’ 180 Kd GP |
| ovarian | CA 19-9 or GICA |
| ovarian | ‘PLAP’ 67 Kd GP |
| ovarian | 72 Kd |
| ovarian | 69 Kd PLAP |
| ovarian | Unknown Mᵣ PLAP |
| ovarian | p185^{HER2} |
| uterus ovary | HMFG-2 |
| GY | HMFG-2 |
| B: BREAST | 330-450 Kd GP |
| | NS |
| | 37 kD |
| | NS |
| | NS |
| | 47 Kd GP |
| | High Mᵣ GP |
| | High Mᵣ GP |
| | NS |
| | NS |
| | 1 (Ma) blood group Ags |
| | NS |
| | oestrogen receptor |
| | EGF Receptor |
| | Laminin Receptor |
| | erb B-2 p185 |
| | NS |
| | 126 Kd GP |
| | NS |
| | NS |
| | 95 Kd |
| | 100 Kd |
| | NS |
| | 24 Kd |
| | 90 Kd GP |
| | CEA & 180 Kd GP |
| | colonic & pancreatic mucin similar to Ca |
| | 19-9 |
| | milk mucin core protein |
| | milk mucin core protein |
| | affinity-purified milk mucin |
| | p185^{HER2} |
| | CA 125 >200 Kd GP |
| | High Mᵣ mucin/glycolipid |
| | High Mᵣ mucin |
| | ‘gp48’ 48 Kd GP |
| | 300-400 Kd GP |
| | ‘TAG-72’ high Mᵣ mucin |
| | ‘CEA’ 180 Kd GP |
| | ‘PLAP’ 67 Kd GP |
| | HMFG-2 >400 Kd GP |
| | NS |
| C: COLORECTAL | TAG-72 High Mᵣ mucin |
| | GP37 |
| | Surface GP |
| | CEA |
| | CEA |
| | cell surface AG |
| | secretory epithelium |
| | surface glycoprotein |
| | NS |
| | NS |
| | NS |
| | cell membrane & cytoplasmic Ag |
| | CEA & vindesine |
| | gp72 |
| | high Mᵣ mucin |
| | high Mᵣ mucin |
| | CEA 180 Kd GP |
| | 60 Kd GP |
| | CA-19-9 (or GICA) |
| | Lewis a |
| | Lewis a |
| | colonic mucus |
| D: MELANOMA | p97^{a} |
| | p97^{a} |
| | p97^{b} |
| | p97^{c} |
| | p97^{c} |
| | p97^{d} |
| | p97^{e} |
| | p155 |
| | G_{D3} disialogan-glioside |
| | p210, p60, p250 |
| | p280 p440 |
| | GP 94, 75, 70 & 25 |
| | P240-P250, P450 |
| | 100, 77, 75 Kd |
| | 94 Kd |
| | 4 GP chains |
| | GP 74 |
| | GP 49 |
| | 230 Kd |
| | 92 Kd |
| | 70 Kd |
| | HMW MAA similar to 9·2·27 AG |
| | HMW MAA similar to 9·2·27 AG |
| | GP95 similar to 376·96S 465·12S |
| | GP125 |
| | CD41 |
| E: GASTROINTESTINAL | high Mᵣ mucin |
| Pancreas, stomach | |
| gall bladder, pancreas, | high Mᵣ mucin |
| stomach | |
| Pancreas | NS |
| Pancreas, stomach, | ‘TAG-72’ high Mᵣ mucin |
| oesophagus | |
| Stomach | ‘CEA’ 180 Kd GP |
| Pancreas | HMFG-2 >400 Kd GP |
| G.I. | NS |
| Pancreas, stomach | CA 19-9 (or GICA) |
| Pancreas | CA125 GP |
| F: LUNG | p185^{HER2} |
| non-small cell lung carcinoma | |
| | high Mᵣ mucin/glycolipid |
| | ‘TAG-72’ high Mᵣ mucin |
| | high Mᵣ mucin |
| | ‘CEA’ 180 kD GP |
| Malignant Gliomas | cytoplasmic antigen from 85HG-22 cells |
| | cell surface Ag from 85HG-63 cells |
| | cell surface Ag from 86HG-39 cells |
| | cell surface Ag from 86HG-39 cells |
| G: MISCELLANEOUS | p53 |
| small round cell tumors | neural cell adhesion molecule |
| Medulloblastoma | |
| neuroblastoma | |
| rhabdomyosarcoma | |
| Neuroblastoma | |
| renal cancer & glioblastomas | p155 |
| Bladder & laryngeal cancers | “Ca Antigen” 350-390 kD |
| Neuroblastoma | GD2 |
| Prostate | gp48 48 kD GP |
| Prostate | 60 kD GP |
| Thyroid | ‘CEA’ 180 kD GP |

2. Immunomodulators

In another aspects of the invention, it is contemplated that the insect cell composition may further comprise a therapeutically effective composition of an immunomodulator. It is envisioned that an immunomodulator would constitute a cytokine, hematapoietin, colony stimulating factor, interleukin, interferon, growth factor or combination thereof. As used herein certain embodiments, the terms “cytokine” are the same as described in U.S. Pat. No. 5,851,984, incorporated herein by reference in its entirety, which reads in relevant part:

The term cytokine is a generic term for proteins released by one cell population which act on another cell as intercellular mediators. These proteins may also act on the producing cells in an autocrine manner. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-.alpha. and -.beta; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-.alpha. and TGF-.beta.; insulin-like growth factor-I and -II; erythropoietin (EPO); osteoinductive factors; interferons such as interferon-α, -.β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1.alpha., IL-2, IL-3, IL-4, IL-5, IL-6, IL-7, IL-8, IL-9, IL-11, IL-12; IL-13, IL-14, IL-15, IL-16, IL-17, IL-18, LIF, G-CSF, GM-CSF, M-CSF, EPO, kit-ligand or FLT-3. As used herein, the term cytokine includes proteins from natural sources or from recombinant cell culture and biologically active equivalents of the native sequence cytokines.

a. β-interferon

β-interferon (IFN-β) is low molecular weight protein that is produced by many cell types, including epithelial cells, fibroblasts and macrophages. Cells that express endogenous IFN-β are resistant to viral infection and replication. The β-interferon genes from mouse (GenBank accession numbers X14455, X14029) and human (GenBank accession numbers J00218, K00616 and M11029) have been isolated and sequenced. IFN-β is a multifunctional glycoprotein that can inhibit tumor growth both directly, by suppressing cell replication and inducing differentiation or apoptosis and indirectly by activating tumoricidal properties of macrophages and NK cells, by suppressing tumor angiogenesis and by stimulating specific immune response.

b. Interleukin-2

Interleukin-2 (IL-2), originally designated T-cell growth factor I, is a highly proficient inducer of T-cell proliferation and is a growth factor for all subpopulations of T-lymphocytes. IL-2 is an antigen independent proliferation factor that induces cell cycle progression in resting cells and thus allows clonal expansion of activated T-lymphocytes. Since freshly isolated leukemic cells also secrete IL-2 and respond to it IL-2 may function as an autocrine growth modulator for these cells capable of worsening ATL. IL-2 also promotes the proliferation of activated B-cells although this requires the presence of additional factors, for example, ILA. In vitro IL-2 also stimulates the growth of oligodendroglial cells. Due to its effects on T-cells and B-cells IL-2 is a central regulator of immune responses. It also plays a role in anti-inflammatory reactions, in hematopoiesis and in tumor surveillance. IL-2 stimulates the synthesis of IFN-γ in peripheral leukocytes and also induces the secretion of IL-1 , TNF-α and TNF-β. The induction of the secretion of tumoricidal cytokines, apart from the activity in the expansion of LAK cells, (lymphokine-activated killer cells) are probably the main factors responsible for the antitumor activity of IL-2.

C. GM-CSF

GM-CSF stimulates the proliferation and differentiation of neutrophilic, eosinophilic, and monocytic lineages. It also functionally activates the corresponding mature forms, enhancing, for example, to the expression of certain cell surface adhesion proteins (CD-11A, CD-11C). The overexpression of these proteins could be one explanation for the observed local accumulation of granulocytes at sites of inflammation. In addition, GM-CSF also enhances expression of receptors for fMLP (Formyl-Met-Leu-Phe) which is a stimulator of neutrophil activity.

### F. PHARMACEUTICALLY ACCEPTABLE CARRIERS

Aqueous compositions of the present invention comprise an effective amount of insect cells or insect cell extracts and immunomodulatroy proteins dissolved or dispersed in a pharmaceutically acceptable carrier or aqueous medium. The phrases “pharmaceutically and pharmacologically acceptable” refer to molecular entities or compositions that do not produce an adverse, allergic or other untoward reaction when administered to an animal, or a human as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The active compounds may generally be formulated for administration to a primary tumor site, e.g., formulated for injection. The preparation of an aqueous compositions that contain an effective amount of insect cells or insect cell extracts as an active component or ingredient will be known to those of skill in the art in light of the present disclosure. Typically, such compositions can be prepared as liquid solutions or suspensions; solid forms suitable for using to prepare solutions or suspensions upon the addition of a liquid prior to injection can also be prepared; the preparations can also be emulsified.

The pharmaceutical forms suitable for injection use include sterile aqueous solutions or dispersions; formulations including sesame oil, peanut oil or aqueous propylene glycol; or sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, or mixtures thereof, and in oils. Under ordinary conditions of storage and use, these preparations contain a preservative to prevent the growth of microorganisms.

Insect cells or insect cell extracts of the present invention can be formulated into a composition in a neutral and/or salt form. Pharmaceutically acceptable salts, include the acid addition salts (formed with the free amino groups of the protein) which are formed with inorganic acids such as, for example, hydrochloric and phosphoric acids, and such organic acids as acetic, oxalic, tartaric, mandelic, and the like. Salts formed with the free carboxyl groups can also be derived from inorganic bases such as, for example, sodium, potassium, ammonium, calcium, ferric hydroxides, or such organic bases as isopropylamine, trimethylamine, histidine, procaine and the like. In terms of using peptide as active ingredients, the technology of U.S. Pat. Nos. 4,608,251; 4,601,903; 4,599,231; 4,599,230; 4,596,792; and 4,578,770, each incorporated herein by reference, may be used.

The carrier can also be a solvent or dispersion medium containing, for example, water, ethanol, polyol (for example, glycerol, propylene glycol, and liquid polyethylene glycol, and the like), suitable mixtures thereof, or vegetable oils. The proper fluidity can be maintained, for example, by the use of a coating, such as lecithin, by the maintenance of the required particle size in the case of dispersion, or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars and sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary, and the liquid diluent first rendered isotonic with sufficient saline or glucose. In this connection, sterile aqueous media which can be employed will be known to those of skill in the art in light of the present disclosure. For example, one dosage could be dissolved in 1 ml of isotonic NaCl solution or added to 1000 ml of hypodermoclysis fluid, and injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

The insect cells or insect cell extracts may be formulated within a therapeutic mixture to comprise about 0.0001 to 1.0 milligrams, about 0.001 to 0.1 milligrams, about 0.1 to 1.0 or even about 10 milligrams per dose or so. Multiple doses can also be administered.

In a particular embodiment of the invention, the insect cells or insect cell extract composition may be associated with a lipid. The insect cells or insect cell extract composition associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. The insect cells or insect cell extract composition associated compositions of the present invention are not limited to any particular structure in solution. For example, they may be present in a bilayer structure, as micelles, or with a “collapsed” structure. They may also simply be interspersed in a solution, possibly forming aggregates which are not uniform in either size or shape.

Lipids are fatty substances which may be naturally occurring or synthetic lipids. For example, lipids include the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes.

Phospholipids may be used for preparing the liposomes according to the present invention and may carry a net positive, negative, or neutral charge. Diacetyl phosphate can be employed to confer a negative charge on the liposomes, and stearylamine can be used to confer a positive charge on the liposomes. The liposomes can be made of one or more phospholipids.

A neutrally charged lipid can comprise a lipid with no charge, a substantially uncharged lipid, or a lipid mixture with equal number of positive and negative charges. Suitable phospholipids include phosphatidyl cholines and others that are well known to those of skill in the art.

Lipids suitable for use according to the present invention can be obtained from commercial sources. For example, dimyristyl phosphatidylcholine (“DMPC”) can be obtained from Sigma Chemical Co., dicetyl phosphate (“DCP”) is obtained from K & K Laboratories (Plainview, N.Y.); cholesterol (“Chol”) is obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Preferably, chloroform is used as the only solvent since it is more readily evaporated than methanol.

Phospholipids from natural sources, such as egg or soybean phosphatidylcholine, brain phosphatidic acid, brain or plant phosphatidylinositol, heart cardiolipin and plant or bacterial phosphatidylethanolamine are preferably not used as the primary phosphatide, i.e., constituting 50% or more of the total phosphatide composition, because of the instability and leakiness of the resulting liposomes.

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; or by transfer of liposomal lipids to cellular or subcellular membranes, or vice versa, without any association of the liposome contents. Varying the liposome formulation can alter which mechanism is operative, although more than one may operate at the same time.

In certain embodiments of the invention, the lipid may be associated with a hemagglutinating virus (HVJ). This has been shown to facilitate fusion with the cell membrane and promote cell entry of liposome-encapsulated DNA (Kaneda et al., 1989). In other embodiments, the lipid may be complexed or employed in conjunction with nuclear non-histone chromosomal proteins (HMG-1) (Kato et al., 1991). In yet further embodiments, the lipid may be complexed or employed in conjunction with both HVJ and HMG-1.

Liposomes used according to the present invention can be made by different methods. The size of the liposomes varies depending on the method of synthesis. A liposome suspended in an aqueous solution is generally in the shape of a spherical vesicle, having one or more concentric layers of lipid bilayer molecules. Each layer consists of a parallel array of molecules represented by the formula XY, wherein X is a hydrophilic moiety and Y is a hydrophobic moiety. In aqueous suspension, the concentric layers are arranged such that the hydrophilic moieties tend to remain in contact with an aqueous phase and the hydrophobic regions tend to self-associate. For example, when aqueous phases are present both within and without the liposome, the lipid molecules may form a bilayer, known as a lamella, of the arrangement XY-YX. Aggregates of lipids may form when the hydrophilic and hydrophobic parts of more than one lipid molecule become associated with each other. The size and shape of these aggregates will depend upon many different variables, such as the nature of the solvent and the presence of other compounds in the solution.

Liposomes within the scope of the present invention can be prepared in accordance with known laboratory techniques. In one embodiment, liposomes are prepared by mixing liposomal lipids, in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min. to 2 hours, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

In the alternative, liposomes can be prepared in accordance with other known laboratory procedures: the method of Bangham et al. (1965), the contents of which are incorporated herein by reference; the method of Gregoriadis, as described in DRUG CARRIERS IN BIOLOGY AND MEDICINE, G. Gregoriadis ed. (1979) pp. 287-341, the contents of which are incorporated herein by reference; the method of Deamer and Uster (1983), the contents of which are incorporated by reference; and the reverse-phase evaporation method as described by Szoka and Papahadjopoulos (1978). The aforementioned methods differ in their respective abilities to entrap aqueous material and their respective aqueous space-to-lipid ratios.

The dried lipids or lyophilized liposomes prepared as described above may be dehydrated and reconstituted in a solution of inhibitory peptide and diluted to an appropriate concentration with an suitable solvent, e.g., DPBS. The mixture is then vigorously shaken in a vortex mixer. Unencapsulated nucleic acid is removed by centrifugation at 29,000×g and the liposomal pellets washed. The washed liposomes are resuspended at an appropriate total phospholipid concentration, e.g., about 50-200 mM. The amount of nucleic acid encapsulated can be determined in accordance with standard methods. After determination of the amount of nucleic acid encapsulated in the liposome preparation, the liposomes may be diluted to appropriate concentrations and stored at 4° C. until use.

A pharmaceutical composition comprising the liposomes will usually include a sterile, pharmaceutically acceptable carrier or diluent, such as water or saline solution.

### G. THERAPIES

1. Treatment of Non-Brain Tumors

In accordance with the present invention, one aspect of the claimed method will involve the administration of the insect cell-immunomodulator of the present invention to a tumor site. The methods of administration may vary depending upon the type of tumor and its location with respect to other organs and tissues. Those of skill in the art will be aware of the various techniques to achieve appropriate contact.

By way of example, the following methods may be employed. First, one may utilize the tumor's vasculature to deliver the composition. Intraarterial or intravenous injection of the composition can target various parts of the tumor, including those that may be remote to a site of access. Second, one may employ direct injection of the tumor. Multiple injections around the edge of the tumor (circumferential) may be used. Multiple deep injections into the tumor body can also be employed. Third, one may utilize partial resection to expose various portions of the tumor or to create a “pocket” into which the composition may be introduced. In a particular embodiment, one may use continuous perfusion/infusion of the tumor or tumor bed. This may have the added advantage of increased exposure to immune cells. Multiple injections over time may achieve the same effect. Fourth, one may mix the composition with a resected tumor tissue that has or has not been irradiated, treated with chemotherapeutic agents, or other ex vivo manipulations. One may then inject the mixtures into the subcutis or other tissues as a tumor vaccine.

2. Combination Therapies

In order to increase the efficacy of a cancer therapy, it may be desirable to combine more than one therapeutic approach in the treatment of hyperproliferative disease. More generally, these other compositions would be provided in a combined amount effective to kill or inhibit proliferation of the cell. This process may involve subjecting the subject to both therapies at the same time. Alternatively, one therapy may precede or follow the other therapy by intervals ranging from minutes to weeks. Generally, one would ensure that a significant period of time did not expire between the time of each therapy such that both therapies would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations. Various combinations may be employed, where the insect cell therapy-immunomodulator is “A” and the secondary agent, such as radio-, chemo-, gene therapy or surgery is “B”:

| | | | | |
|---|---|---|---|---|
| A/B/A | B/A/B B/B/A | A/A/B | A/B/B B/A/A | A/B/B/B B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, fanesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Genes

In yet another embodiment, the secondary treatment is a secondary gene therapy in which a second therapeutic polynucleotide is administered before, after, or at the same time a first therapeutic polynucleotide encoding all of part of an MDA-7 polypeptide. Delivery of a vector encoding either a full length or truncated MDA-7 in conduction with a second vector encoding one of the following gene products will have a combined anti-hyperproliferative effect on target tissues. Alternatively, a single vector encoding both genes may be used. A variety of proteins are encompassed within the invention, some of which are described below.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al, 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primnary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, M-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis. (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{w}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Hormonal Therapy

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

3. Kits

Therapeutic or prophylactic kits of the present invention are kits comprising insect cells or insect cell extract composition comprising immunomodulatory proteins. Such kits will generally contain, in suitable container means, a pharmaceutically acceptable formulation of insect cells or insect cell extract composition in a pharmaceutically acceptable formulation. The kit may have a single container means, or it may have distinct container means for each compound.

When the components of the kit are provided in one or more liquid solutions, the liquid solution is an aqueous solution, with a sterile aqueous solution being particularly preferred. The insect cells or insect cell extract composition may also be formulated into a syringeable composition. In which case, the container means may itself be a syringe, pipette, or other such like apparatus, from which the formulation may be applied to an infected area of the body, injected into an animal, and even applied to or mixed with the other components of the kit.

However, the components of the kit may be provided as dried powder(s). When reagents or components are provided as a dry powder, the powder can be reconstituted by the addition of a suitable solvent. It is envisioned that the solvent may also be provided in another container means.

The container means will generally include at least one vial, test tube, flask, bottle, syringe or other container means, into which the insect cells or insect cell extract composition formulation are placed, preferably, suitably allocated. The kits may also comprise a second container means for containing a sterile, pharmaceutically acceptable buffer or other diluent.

The kits of the present invention also will typically include a means for containing the vials in close confinement for commercial sale, such as, e.g., injection or blow-molded plastic containers into which the desired vials are retained.

Irrespective of the number or type of containers, the kits of the invention may also comprise, or be packaged with, an instrument for assisting with the injection/administration or placement of the ultimate insect cells or insect cell extract composition within the body of an animal. Such an instrument may be a syringe, pipette, forceps, and any such medically approved delivery vehicle.

### H. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Mice. Specific pathogen-free female C3H/HeN mice were purchased from the Animal Production Area of the National Cancer Institute-Frederick Cancer Research Facility (Frederick, Md.). The animals were maintained in facilities approved by the American Association for Accreditation of Laboratory Animal Care and in accordance with current regulations and standards of the United States Department of Agriculture, Department of Health and Human Services, and National Institutes of Health. The mice were used in accordance with institutional guidelines when they were 6 to 8 weeks of age, except where otherwise indicated.

Baculovirus, Insect Cells, and Culture Conditions. Grace's medium, wild-type baculovirus, pBlueBacHis2A baculovirus transfer vector, liposome-mediated transfection kit, and Sf9 and High Five (H5) insect cells were purchased from Invitrogen Corporation (Carlsbad, Calif.). Fetal bovine serum (FBS) was purchased from M. A. Bioproducts (Walkersville, Md.), and EXCELL-400 medium from JRH Biosciences (Denver, Colo.). Sf9 and H5 cells were maintained as monolayer cultures in complete TNM-FH medium (Grace's medium supplemented with 10% FBS and Grace's medium supplements) and serum-free medium EXCELL 400, respectively, at 27° C. in an unhumidified chamber. The insect cells and preparations containing H5 cells, baculovirus, and/or IFN-β were free of endotoxins as determined by the Limulus amebocyte lysate assay (Associates of Cape Cod, Woods Hole, Mass.).

Expression of IFN-β. in H5 Insect Cells. Vectors were constructed and expression of IFN-β-induced using a kit from Invitrogen following the manufacturer's instructions as detailed in our previous study (Lu et al., 2002). Briefly, the full coding sequence of murine IFN-β cDNA was subcloned into the baculovirus transfer vector pBlueBacHis2A to derive the recombinant vector pHis2AIFN-β. Recombinant baculovirus encoding the IFN-β. (BVIFN-β) gene was produced by cotransfecting SF9 cells with pHis2AIFN-β and linearized Bac-N-Blue baculovirus DNA by using a liposome-based transfection kit. The recombinant virus was propagated in SF9 cells to achieve 5×10⁸ PFU/ml. To prepare H5BVIFN-β, H5 cells were infected with 3 multiplicities of infection (MOI) of BVIFN-β for 48 h, which led to an accumulation of 2×10⁴ units of IFN-β per 10⁶ H5 cells (determined by Access Biomedical Research Laboratories, Inc., San Diego, Calif.). One unit of H5BVIFN-β contained 2×10⁴ units of IFN-β, 1×10⁶ H5 cells, and 2×10⁷ PFU of BV.

Tumor Models and Immunotherapy. The UV-2237M tumor cell line was derived from a spontaneous lung metastasis produced by parental WV-2237 fibrosarcoma cells originally induced in a C3H/HeN mouse by ultraviolet (UV)-B radiation (Raz, et al., 1981). The K-1735M2 melanoma cell line was derived from spontaneous lung metastases produced by parental K-1735 melanoma cells originally induced in a C3H/HeN mouse by UV-B radiation followed by croton oil painting (Kripke, 1979; Talmadge and Fidler, 1982). UV-2237M or K-1735M2 (2×10⁵, unless otherwise indicated) cells were inoculated s.c. into syngeneic C3H/HeN mice. When tumors reached 4-5 mm in diameter, the lesions were injected with phosphate-buffered saline (PBS) or H5BVIFN-β. The tumor size in 2 perpendicular diameters was measured with calipers every 5-7 days. Non-palpable lesions were considered to have been eradicated.

Experimental Brain Metastasis. Suspensions of UV-2237M or K-1735M2 cells were injected into the internal carotid artery of C3H/HeN mice using the technique described previously (Schackert and Fidler, 1988). The mice were killed when they were moribund or up to 180 days after the injection of the tumor cells. The brains were removed and fixed in 10% buffered formalin solution. Each brain was serially sectioned. The tissues were stained with hematoxylin and eosin and examined for the presence of metastases.

Induction of Long-term Tumor-specific Immunity and Intracarotid Challenge. Six weeks after eradication of s.c. UV-2237M or K-1375 M2 tumors (by intralesional injection of H5BVIFN-β), C3H/HeN mice were divided into 2 groups. The mice were challenged by intracarotid injection with TV-2237M cells or with K-1735M2 cells. Naive C3H mice injected with either cell line served as controls. Mice were killed when they were moribund, and the brains were harvested for histological examination. C3H/HeN mice were inoculated s.c. with UV-2237M or K-1735M2 cells. Two weeks later, all mice developed s.c. tumors averaging 7-8 mm in diameter. The mice were anesthetized with Nembutol, and the s.c. tumors were resected. The mice received intracarotid injections of either UV-2237M cells or K-1735M2 cells. Naïve C3H/HeN mice injected with tumor cells in the internal carotid artery served as controls. The mice were killed when they became moribund, and the brains were harvested for histologic examination.

Therapy of Occult Brain Metastases. The inventors determined whether the injection of H5BVIFN-β into a subcutaneous UV-2237M tumor generated an immune rejection of brain metastasis. Mice were implanted s.c. with 2×10⁵ UV-2237M cells in the right flank. When the s.c. tumors reached 3-5 mm in diameter (day 7), the mice were divided into 2 groups to receive an internal carotid artery injection of 2×10⁴ UV-2237M cells or 2×10⁴ K-1735M cells. Two days later, the TV-2237M s.c. tumors were injected with either lyophilized H5BVIFN-β in 100 μl PBS or with 100 μl PBS. The mice were observed daily. Subcutaneous tumors exceeding 15 mm in diameter were resected. The mice were killed when moribund and autopsied. The brains were fixed in 10% fonnalin and examined histologically for the presence of brain metastasis.

Depletion of T Cells. One day before and one and two days after the intratumoral injection of H5BVIFN-β, mice were injected i.p. with rat monoclonal antibodies (mAb) against CD4 (GK1.5 mAb, American Type Culture Collection, 200 μg/mouse), CD8 (GK1.5 mAb, American Type Culture Collection, 200 μg/mouse), or CD4 plus CD8. Control mice received 3 i.p. injections of rat IgG (200 μg/mouse). In control experiments, 3 i.p. injections of anti-CD4 and anti-CD8 mAb produced a 75% and a 90% reduction of CD4+ and CD8+ T cells, respectively, in the spleens as indicated by flow cytometric analysis. The depletion persisted for up to 5 weeks.

Immunohistochemistry. immunohistochemical analyses of tumor tissues were performed as described previously (Lu et al., 1999). Briefly, at necropsy, tumor tissues were cut into 5 mm pieces, placed in OCT compound (Miles Laboratories, Elkhart, Ind.), and snap-frozen in liquid nitrogen. Frozen sections (8-10 μm) were fixed in cold acetone and treated with 3% hydrogen peroxide in ethanol (v/v). The treated slides were blocked in PBS containing 5% normal horse serum/1% normal goat serum and incubated with antibodies to CD4 (American Type Culture Collection), or CD8 (PharMingen, San Diego, Calif.) antigen for 18 h at 4° C. in a humidified chamber. The sections were rinsed and incubated with peroxidase-conjugated secondary antibodies. A positive reaction was visualized by incubating the slides with stable DAB (Research Genetics, Huntsville, Ala.) and counterstained with Mayer's hematoxylin (Research Genetics). The slides were dried and mounted with Universal mount (Research Genetics). The images were digitized using a Sony 3CD color video camera,(Sony Corporation, Tokyo, Japan) and a personal computer equipped with Optimas image analysis software (Optimas Corporation, Bothell, Wash.). For imnmunohistochemical staining using an antibody against proliferating cell nuclear antigen (PCNA), paraffin sections (3-5 μm) of the tumor samples were placed on ProbeOn slides (Fischer Scientific) and stained as described for the frozen sections after deparaffinization and rehydration.

Statistical Analysis. Survival estimates and median survivals were determined using the method of Kaplan and Meier (Kaplan and Meier, 1958). The survival data were tested for significance using a logrank test. The significance of differences in tumor incidence and tumor size was analyzed by the χ² test and ANOVA, respectively.

### Example 2

### Results (Brain Metastasis)

Eradication of s.c. Tumors by H5BVIFN-β Confers Tumor-specific Immune Protection against Brain Metastasis. C3H/HeN mice were implanted s.c. with either UV-2237M or K-1735M2 cells, and on day 7, the resulting tumors were injected with H5BVIFN-β. Six weeks after the complete regression of the UV-2237M fibrosarcoma or K-1735M2 melanoma (which was 9-10 weeks after injection), the mice were randomized to receive an intracarotid injection of either UV-2237M or K-1735M2 cells. In naive (control) mice, brain metastases developed in 9/10 and 9/9 mice, with a median survival of 27 and 23 days, respectively (Table 1). Mice cured of s.c. UV-2273M tumors by intralesional injection of H5BVIFN-β did not develop UV-2237M brain metastases but did develop K-1735M2 brain metastases. The median survival of these two groups of mice was >180 days and 18 days, respectively (P<0.001). Similarly, 5 of 7 mice cured of s.c. K-1735M2 melanoma did not develop brain metastases of K-1735M2 cells but did develop brain metastases of the UV-2237M fibrosarcoma (6 of 7 mice). The median survival of these mice was >180 days and 30 days, respectively (p<0.001).

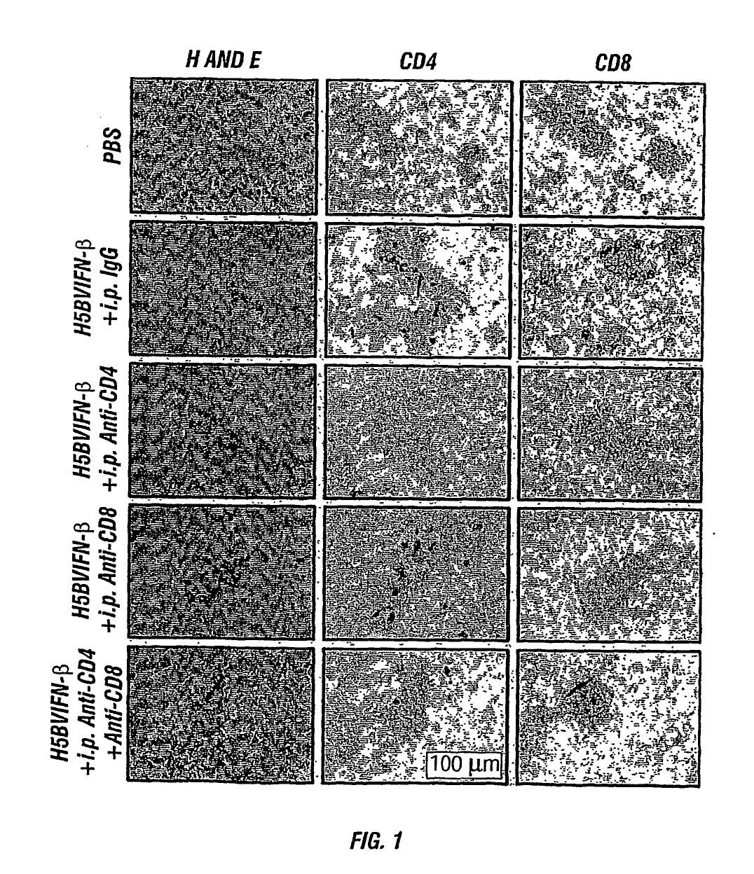

The mere growth of tumors in the subcutis did not confer systemic immunity. Mice whose s.c. tumors were surgically excised (rather than treated with H5BVIFN-β) were challenged with tumor cells injected into the internal carotid artery. Brain metastases of UV-2237M or K-1735M2 cells developed in 8 of 10 and 5 of 5 mice originally implanted s.c. with UV-2237M tumors. Median survival of the mice was 31 and 22 days, respectively (Table 2). Similarly, the surgical removal of s.c. K1735M2 tumors did not significantly alter the development of brain metastasis by UV-2237M or K-1735M2 cells (Table 2). The growth of TV-2237M and K-1735M2 tumors was confirmed by histological analysis. Images of a typical histological staining are shown in FIG. 1, demonstrating that the intracarotid injection of UV-2237M or K-1735M2 cells produced tumors in control mice, but not in mice cured of s.c. UV-2237M or K-1735M2 tumors by injection of H5BVIFN-β.

TABLE 2
Specific inhibition of brain metastasis by injection of H5BVIFN-β into subcutaneous growing neoplasms

| | Intracarotid injection of: | | | |
|---|---|---|---|---|
| | UV-2237M cells | | K-1735M2 cells | |
| Treatment of subcutaneous | Incidence of brain | Median (Range) | Incidence of brain | Median (Range) Survival in |
| tumor | metastasis | Survival in days | metastasis | days |
| No tumor | 9/10 | 27 (2->180) | 9/9 | 23 (20-82) |
| UV-2237M-resected | 8/10 | 31 (24->180) | 5/5 | 22 (20-26) |
| UV-2237M-H5BVIFN-β | 0/15* | >180 | 5/5 | 18 (17-39) |
| K-1735M2-resected | 5/5 | 27 (21-31) | 7/9 | 23 (20->180) |
| K-1735M2-H5BVIFN-β | 6/7 | 30 (18->180) | 2/7* | >180 (40->180)* |
| C3H/HeN mice were injected s.c. with UV-2237-M or K-1375M2 cells. The H5BVIFN-β preparation was injected into the tumors on day 7 (when the tumors reached the size of 4-5 mm in diameter). In some mice, uninjected tumors were resected on day 12 (when the tumors reached 7-8 mm in diameter). Six weeks after s.c. tumor regression (H5BVIFN-β) or resection, the mice were injected in the | | | | |
| # internal carotid artery with either UV-2237M or K-1735M2 cells. Mice were killed when they were moribund. Surviving mice were killed on day ≧180. The brains were harvested for histologic examination. | | | | |

*P < 0.001.

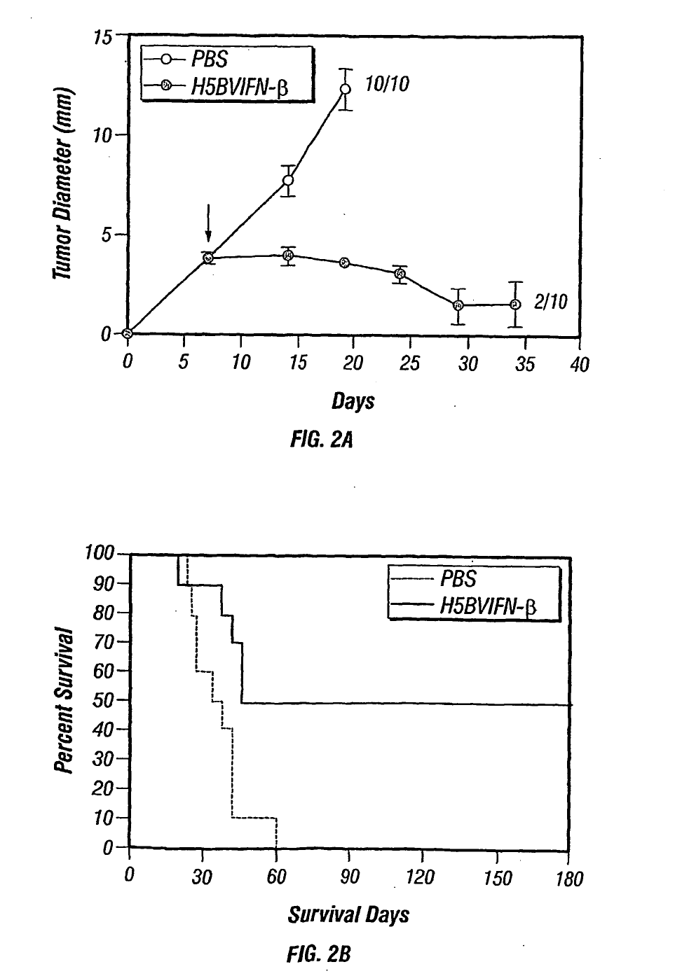

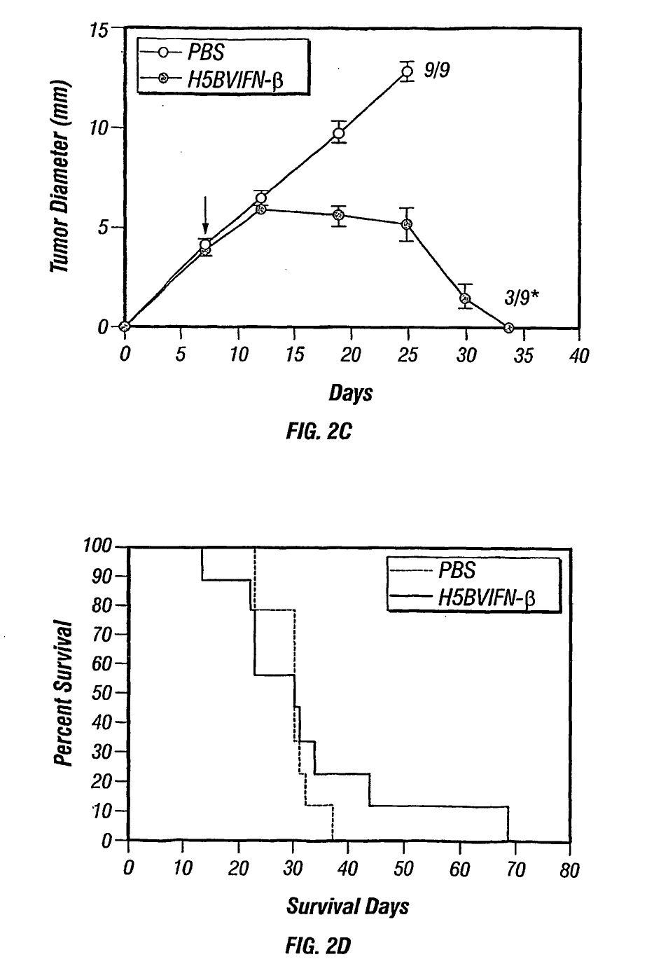

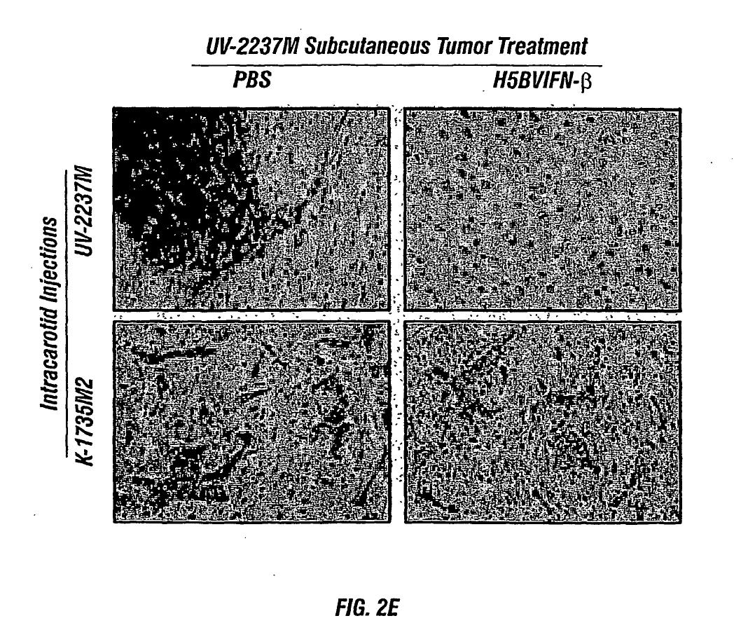

Eradication of Established s.c. Tumors and Occult Brain Metastasis by H5BVIFN-β Therapy. Next, the inventors determined whether the injection of H5BVIFN-β into s.c. tumors could eradicate pre-existing, occult brain metastases. First, UV-2237M cells were inoculated s.c. into syngeneic C3H/HeN mice. When the tumors reached 3-5 mm in diameter, the mice were injected in the internal carotid artery with UV-2237M or K-1735M2 cells. Two days later, the s.c. tumors were injected with PBS or 2 units of H5BVIFN-β. The data summarized in FIGS. 2A-E show that a single injection of H5BVIFN-β into the s.c. UV-2237M tumors led to complete regression of the s.c. tumors in 60-80% of mice (FIG. 2A and FIG. 2C) and prolonged the survival of mice with UV-2237M brain metastases (P<0.05, FIG. 2B), but not the survival of mice with K-1735M2 brain metastases (FIG. 2D). Histological examination of the brain confirmed that the injection of H5BVIFN-β into the s.c. tumors eradicated UV-2237M but not K-1735M2 tumors (FIG. 2E).

Eradication of Brain Metastases is Mediated by Both CD4+ and CD8+ Cells. Since the inventors have demonstrated that the eradication of s.c. tumors by H5BVIFN-β therapy requires both CD4+ and CD8+ cells (Lu et al., 2002), the inventors determined whether these T lymphocyte subsets were also involved in the destruction of UV-2237M brain metastases. C3H/HeN mice were injected s.c. with UV-2237M cells. When the resulting tumors reached 5-6 mm in diameter (day 7), the mice were injected in the carotid artery with UV-2237M cells. Two days later (day 9), the mice were injected i.p. with 200 μg/mouse of anti-CD4 and/or anti-CD8 antibodies. The i.p. injections were repeated on days 11 and 13. The s.c. tumors were injected once with the H5BVIFN-β preparation on day 10. Control mice whose s.c. tumors were treated with PBS had a median survival of 36 (29-56) days. Mice injected i.p. with PBS and H5BVIFN-β or IgG and H5BVIFN-β had a median survival of 115 (33-180) days and 180 (33-180) days, respectively.

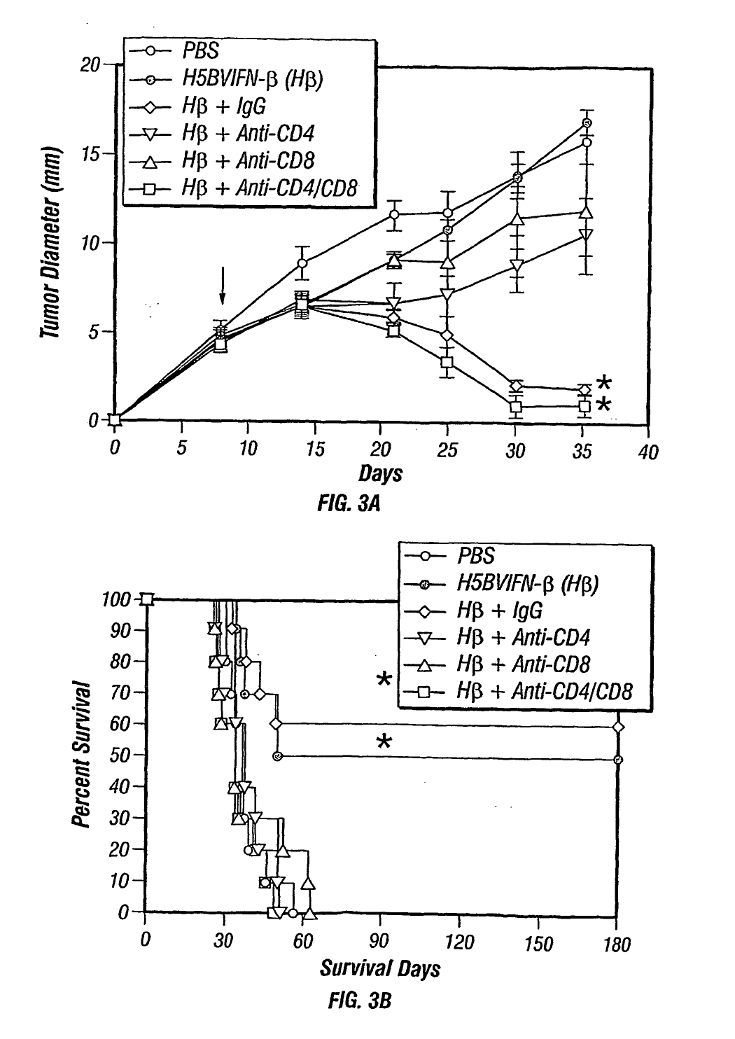

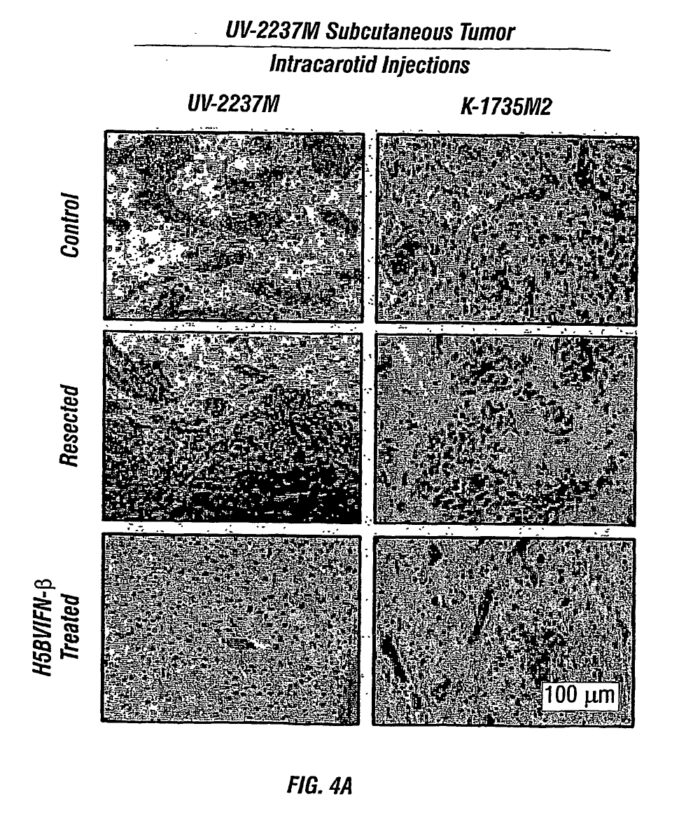

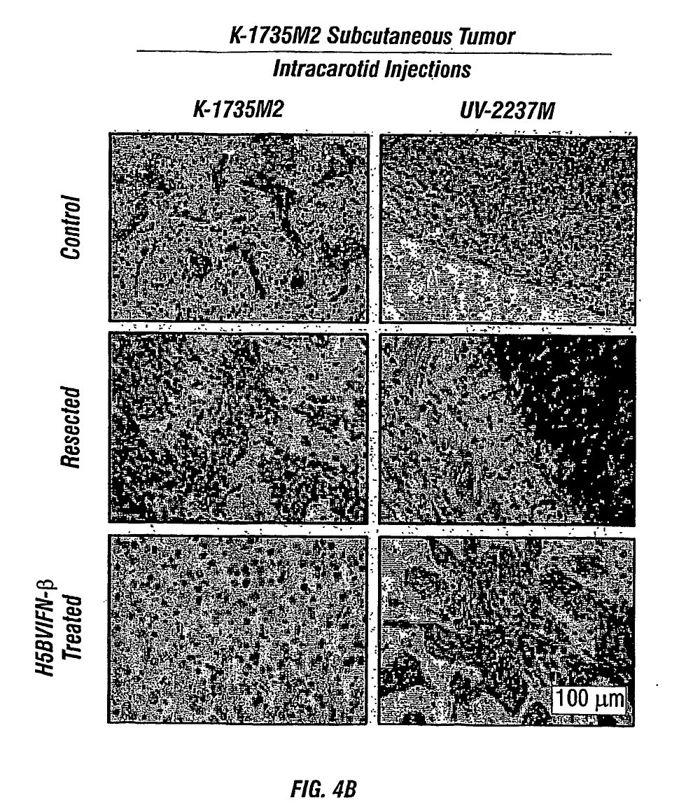

Surviving mice were killed on day 180, and the mice (Fidler et al., 1999) treated with PBS plus H5BVIFN-β and 5 of 6 mice with control IgG plus H5BVIFN-β were histologically free of any brain metastases (P<0.001). In sharp contrast, the median survival of mice injected with anti-CD4 antibody was 37 (31-51) days; with anti-CD8 antibody, 33 (27-61) days; and with anti-CD4 plus anti-CD8, 33 (25-49) days (P<0.001). FIGS. 3A-B. These data suggest that both CD4+ and CD8+ T cells are involved in H5BVIFN-β activity against the UV-2237M tumors in the brain of mice. Immunohistochemical analyses of brain metastases strengthened this suggestion. To determine whether brain metastases were infiltrated by CD4+ and/or CD8+ cells, mice were killed on day 17 of the experiment, i.e., 7 days after the injection of H5BVIFN-β preparation into s.c. tumors. The brains were frozen and examined histologically (FIGS. 4A-B). In control mice, the brain metastases contained numerous CD4+ and CD8+ cells. In mice injected with H5BVIFN-β and IgG, the brain metastases were densely infiltrated by CD4+ and CD8+ cells. These metastases eventually regressed. In mice injected with H5BVIFN-β and antibodies against CD4 and/or CD8 antigens, the number of infiltrating CD4+ or CD8+ cells was significantly reduced. The median survival of mice given anti-CD4 and/or anti-CD8 antibodies did not exceed that of mice that did not receive H5BVIFN-β treatment.

### Example 3

### Results (Lung Metastasis)

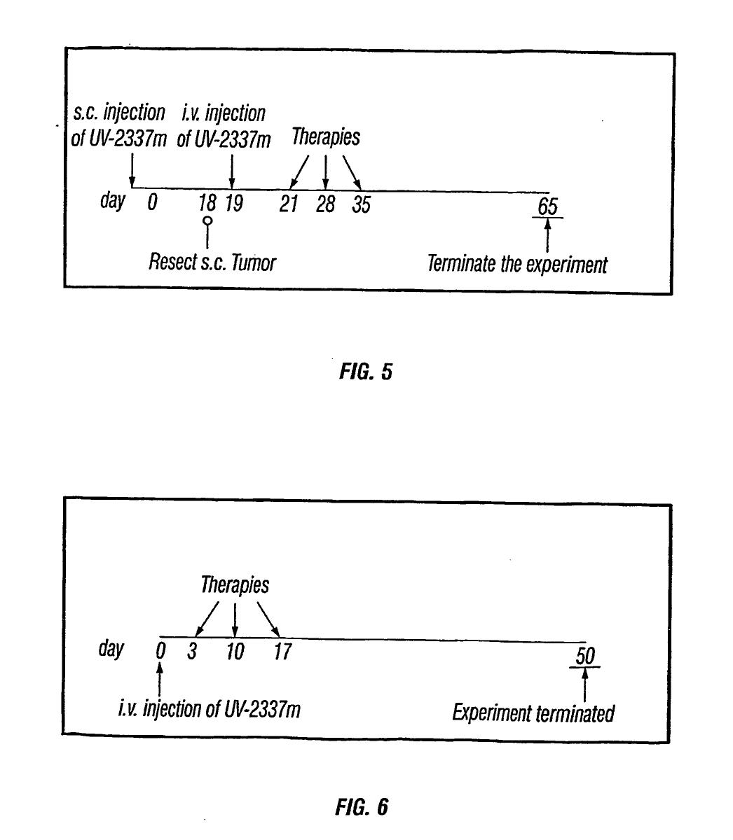

Methods: The effects of subcutaneous injection of a mixture of H5BVIFN-β and irradiated UV-2237m tumor preparation on growth of existing lung metastases in mice with surgically removed s.c. tumors were examined. UV-2237m cells (2×10⁵/mouse) were s.c. injected into 20 C3HVHeN mice. On day 18 after tumor cell inoculation, the tumor-bearing mice were i.v. injected with 5×10⁴/mouse of UV-2237m cells. Five naïve mice were i.v. injected with UV-2237m cells as a control. One day later, the subcutaneous tumors were surgically resected, enzymatically dissociated, and irradiated (2,000 rads from the Cesium-137 source). On day 21, mice in which s.c. tumor were surgically removed were randomized into 4 groups and s.c. injected with PBS, 2×10⁶ lyophilized H5BVIFN-β, 5×10⁶ irradiated cells from UV-2237m tumors, or a mixture of H5BVIFN-β and 5×10⁶ irradiated cells. The treatment was repeated on day 28 and 35 after the subcutaneous tumor cell inoculation. The mice were killed on day 65 (FIG. 5).

TABLE 3

| | Treatment | Lung metastasis, median (range) | | |
|---|---|---|---|---|
| s.c. Tumors | Group | Weight (mg) | Nodules | Incidence |
| No | None | 894 (708-1,820) | 78 (57-83) | 5/5 |
| UV-2237m | PBS | 103 (86-196) | 16 (5-27) | 5/5 |
| UV-2237m | H5BVIFN-β | 111 (49-323) | 21 (7-58) | 5/5 |
| UV-2237m | UV-2237m | 97 (38-608) | 26 (1-63) | 5/5 |
| UV-2237m | UV-2237m + | 3 (0-20) | 1 (0-9) | 3/5 |
| | H5BVIFN-β | | | |

Conclusions: Surgical removal of s.c. UV-2237m tumors significantly suppressed growth of lung metastasis. A therapy with H5BVIFN-β or UV-2237m alone did not affect growth of lung metastasis, but therapy with H5BVIFN-β plus UV-2237m significantly inhibited growth of lung metastasis.

Methods: The effects -of subcutaneous injection of a mixture of H5BVIFN-β and irradiated UV-2237m tumor preparation on growth of existing lung metastases. UV-2237m cells (5×10⁴/mouse) were injected into 40 C3H/HeN mice. On day 3 after the tumor cell inoculation, the mice were randomized into 4 groups and treated by s.c. injection of PBS, 2×10⁶ lyophilized H5BVIFN-β cells, 5×10⁶ irradiated UV-2237m cells (2000 rads from a Cesium-137 source), or H5BVIFN-β plus irradiated UV-2237m cells. The therapy was repeated on days 10 and 17. Mice were killed on day 50 after the i.v. tumor cell inoculation (FIG. 6).

TABLE 4

| | Lung metastasis, median (range) | | |
|---|---|---|---|
| Treatment Group | Weight (mg) | Nodules | Incidence |
| PBS | 763 (246-1206) | 20 (9-31) | 10/10 |
| H5BVIFN-β | 801 (87-1624) | 17 (3-32) | 10/10 |
| UV-2237m | 453 (94-1194) | 12.5 (6-31) | 10/10 |
| H5BVIFN-β + | 376 (0-851) | 8.5 (0-24) | 8/10 |
| UV-2237m | | | |

Conclusions: The therapy with a mixture of lyophilized H5BVIFN-β and irradiated UV-2237m cells did not significantly inhibit growth of UV-2237m lung metastasis.

Methods: C3H/HeN mice were s.c. and i.v. injected with 2×10⁵/mouse of UV2237m cells. On day 7 after the inoculation, s.c. tumors were resected. One day later, the mice were treated by s.c. injection of PBS, a mixture of 2×10⁶ lyophilized H5 cells and 2×10⁴ units of IFN-α, 10⁷ of irradiated UV-2237m cells prepared from subcutaneous tumors, or a mixture of 2×10⁶ lyophilized H5 cells, 2×10⁴ units of IFN-α, and 10⁷ of UV-2237m cells. The treatments were repeated once one week later. The experiment was terminated on day 20 after the therapy (FIG. 11).

TABLE 5

| | | Lung Nodules | |
|---|---|---|---|
| | | Macros-nodules | Micro-nodules |
| Treatment | Lung weight (mg) | (range, median) | (incidence) |
| PBS | 504 ± 193 | (0->200, 65) | 7/8 |
| H5 + IFN-α | 511 ± 133 | (0->200, 85) | 7/8 |
| UV-2237m | 870 ± 296 | (0->200, 75) | 5/6 |
| UV-2237m + | 183 ± 28 | (0-10, 0) | 3/9 |
| H5 + IFN-α | | | |

Conclusions: The results are shown in FIG. 11. Growth of existing lung metastasis was suppressed in mice treated with UV-2237m cells and H5 plus IFN-α, but not with either UV-2237m or H5 plus IFN-α alone.

### Example 4

### Results (INF-α)

Methods: UV-2237m cells (2×10⁵/mouse) were s.c. injected into C3H/HeN mice. On day 7 after tumor cell inoculation, the tumors were injected with PBS or 2×10⁶ lyophilized H5 cells, a mixture of 2×10⁶ lyophilized H5 cells and 1 or 2×10⁴ units of IFN-β or IFN-α. Subcutaneous tumors were measured once a week and the experiment was terminated on day 28 after tumor cell inoculation.

TABLE 6

| Treatment Group | Tumor Incidence |
|---|---|
| PBS | 5/5 |
| Lyophilized H5 cells | 4/5 |
| H5 cells + IFN-β (2 × 10⁴ units) | 5/5 |
| IFN-α (2 × 10⁴ units) | 5/5 |
| H5 cells IFN-α (2 × 10⁴ units) | 2/5 |
| IFN-α (10⁴ units) | 4/5 |
| H5 cells + IFN-α (10⁴ units) | 1/5 |

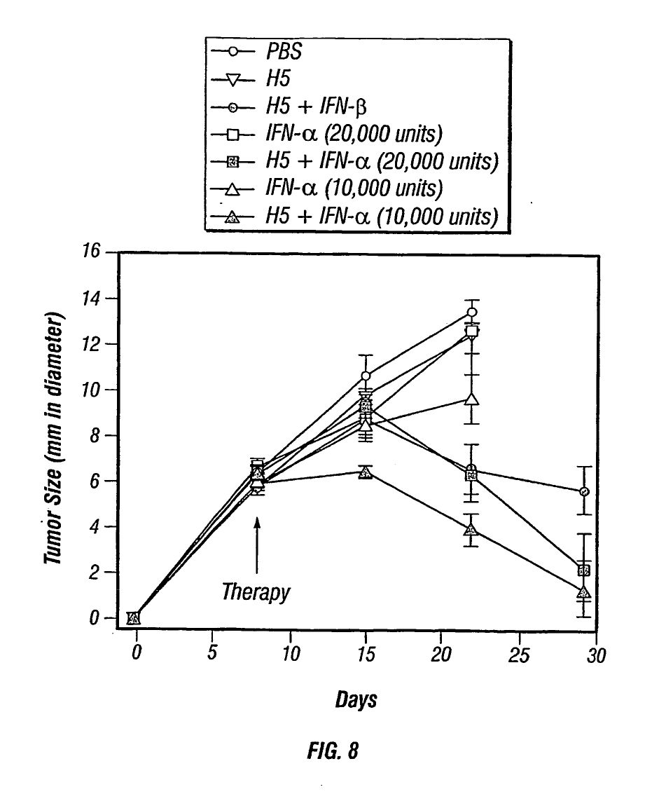

Conclusions: Results are shown in FIG. 8. Intratumoral injection of 1 or 2×10⁴ units of IFN-α alone did not affect growth of UV-2237m tumors in the subcutis of C3H/HeN mice. A therapy using a mixture of IFN-α and lyophilized H5 cells could eradicate UV-2237m tumors in C3H/HeN mice. Treatment of with a mixture of H5 cells and IFN-β failed to eradicate UV-2237m tumors in C3H/HeN mice.

Methods: UV-2237m cells (2×10⁵/mouse) were s.c. injected into 30 C3H/HeN mice. On day 7 after tumor cell inoculation, the tumors were injected with PBS, 2×10⁴ units of IFN-α, 2×10⁴ units of IFN-γ, a mixture of 2×10⁶ lyophilized H5 cells and 2×10⁴ units of IFN-α, or mixture of 2×10⁶ lyophilized H5 cells and 2×10⁴ units of IFN-γ. Subcutaneous tumors were measured once a week and data shown are up to day 28 after tumor cell inoculation.

TABLE 7

| Treatment Group | Tumor Incidence |
|---|---|
| PBS | 5/5 |
| H5 | 5/5 |
| IFN-α | 5/5 |
| IFN-γ | 5/5 |
| H5 + IFN-α | 0/5 |
| H5 + IFN-γ | 4/5 |

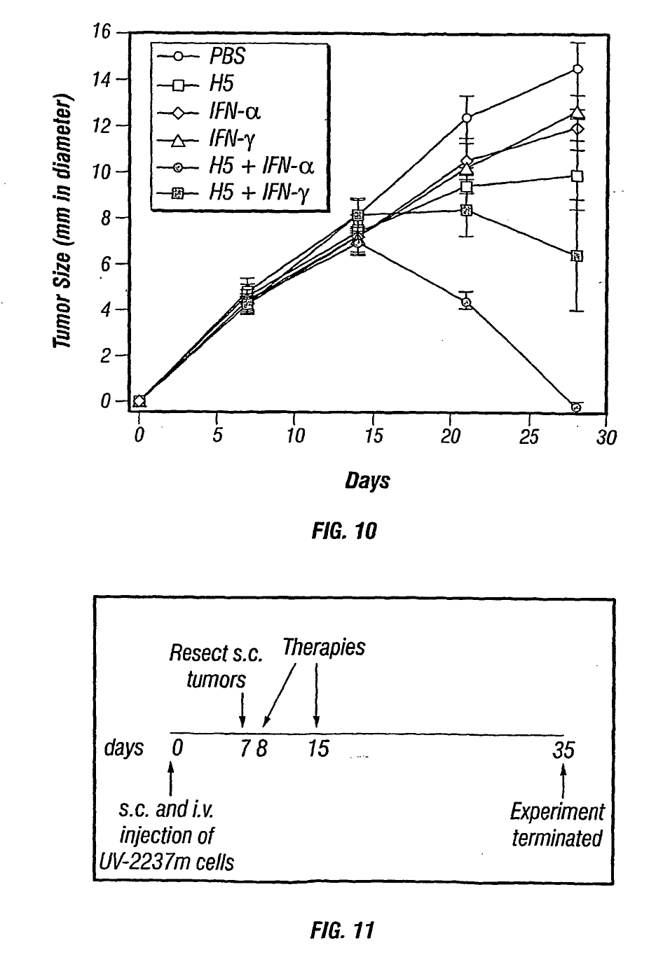

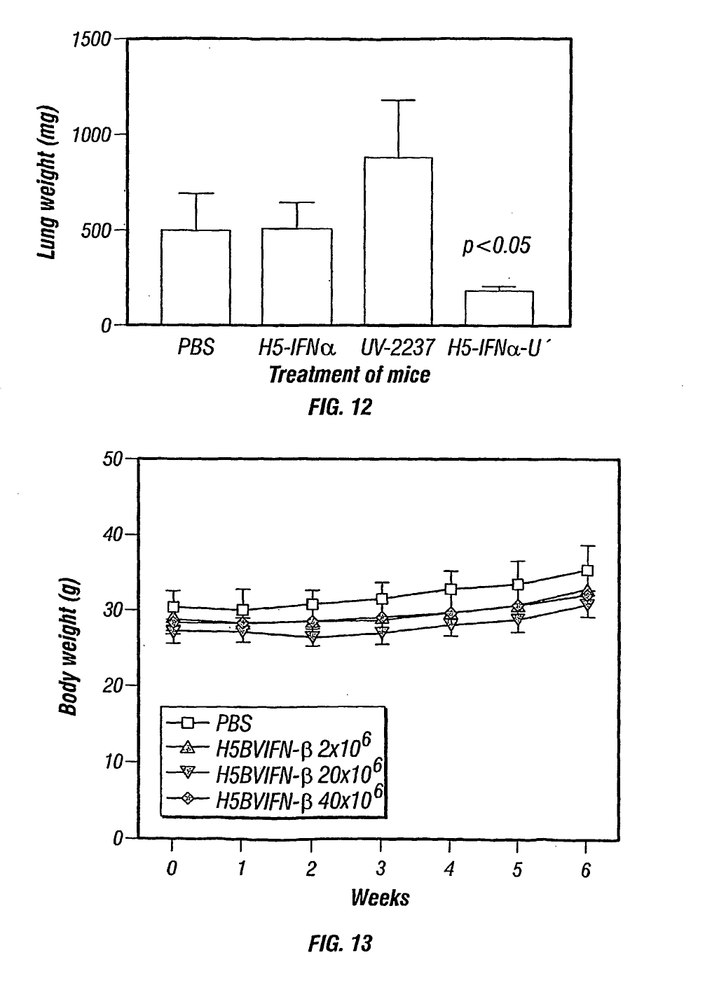

Conclusions: Results are shown in FIG. 10. A therapy with either IFN-α or IFN-γ could not eradicate s.c. UV-2237m tumors. A therapy with a mixture of lyophilized H5 cells and IFN-α eradicated tumors. A therapy with a mixture of lyophilized H5 cells and IFN-γ eradicated s.c. UV-2237m tumor in 1 out of 5 mice and suppressed tumor growth in the rest of mice.

### Example 5

### Results (Components)

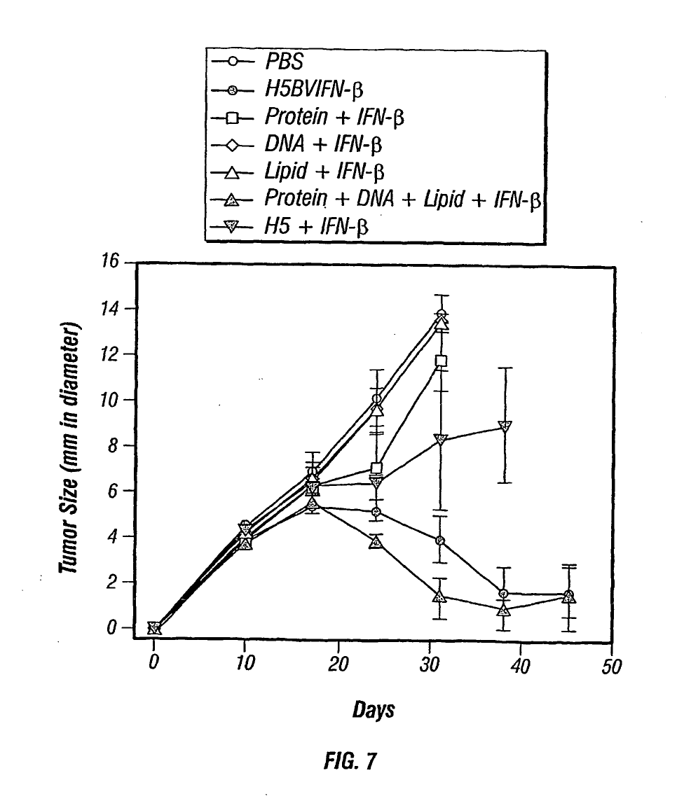

Methods: U-2237m cells (2×10⁵/mouse) were s.c. injected into C3H/HeN mice. On day 7 after tumor cell inoculation, the tumors were injected with PBS or 2×10⁶ lyophilized H5BVIFN-β, a mixture of 2×10⁴ units IFN-β and 2×10⁶ lyophilized H5 cells or components (lipid, protein, and/or DNA) extracted from 2×10⁶ H5 cells. Subcutaneous tumors were measured once a week and the experiment was terminated on day 41 after tumor cell inoculation. Results are shown in FIG. 7.

TABLE 8

| Treatment Group | Tumor Incidence |
|---|---|
| PBS | 5/5 |
| H5BVIFN-β | 2/5 |
| Protein + IFN-β | 3/5 |
| DNA + IFN-β | 5/5 |
| Lipid + IFN-β | 5/5 |
| Protein + DNA + lipids + IFN-β | 1/5 |
| H5 + IFN-β | 4/5 |

Conclusions: In this experiment, the mixture of lyophilized H5 cells and IFN-β failed to eradicate tumors in most mice. However, this was likely due to a change in IFN-β activity, as the IFN-β source was altered. A mixture of IFN-β and DNA/protein/lipid of H5 cells eradicated tumors in 4 out of 5 mice.

Methods: UV-2237m cells (2×10⁵/mouse) were s.c. injected into 35 C3H/HeN mice. Seven days later, the tumors were injected with PBS, 2×10⁶ lyophilized H5BVIFN-β (positive control), a mixture of 2×10⁴ units of IFN-α and 2×10⁶ lyophilized H5 cells, or cellular components (lipid, protein, and/or DNA) extracted from 2×10⁶ H5 cells. Subcutaneous tumors were measured once a week and experiment was terminated on day 29 after tumor cell inoculation.

TABLE 9

| Treatment Group | Tumor Incidence |
|---|---|
| PBS | 5/5 |
| H5BVIFN-β | 0/5 |
| H5 + IFN-α | 1/5 |
| Protein + IFN-α | 5/5 |
| Lipid + IFN-α | 5/5 |
| DNA + IFN-α | 5/5 |
| Protein + lipid + DNA + IFN-α | 3/5 |

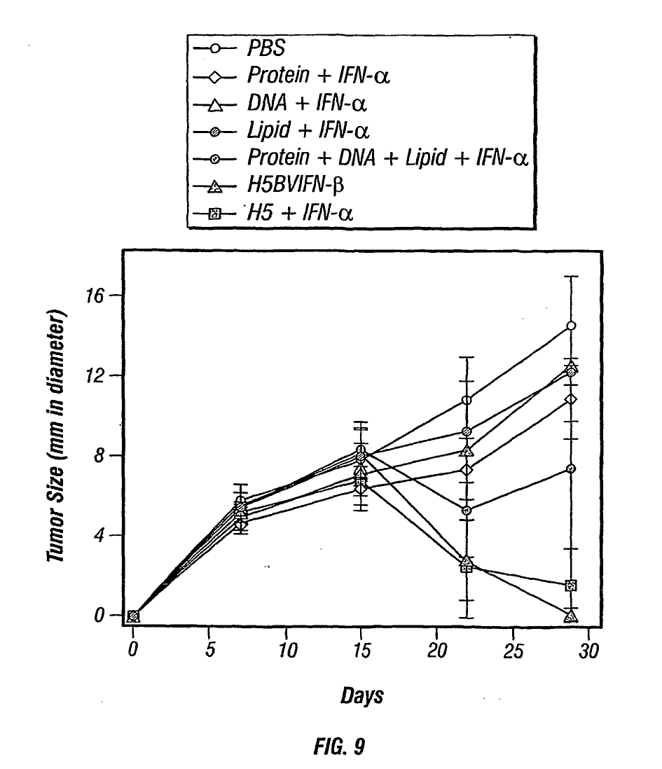

Conclusions: Results are shown in FIG. 9. A therapy with H5BVIFN-β eradicated tumors in 4 out of 5 mice. A therapy with a mixture of lyophilized H5 cells and IFN-α produced similar results as that using H5BVIFN-β. A combination IFN-α and the components of H5 cells was not as effective as those with either H5BVIFN-β or H5 cells plus IFN-α in the therapy against UV2237m tumors.

### Example 6

### Results (Toxicity)

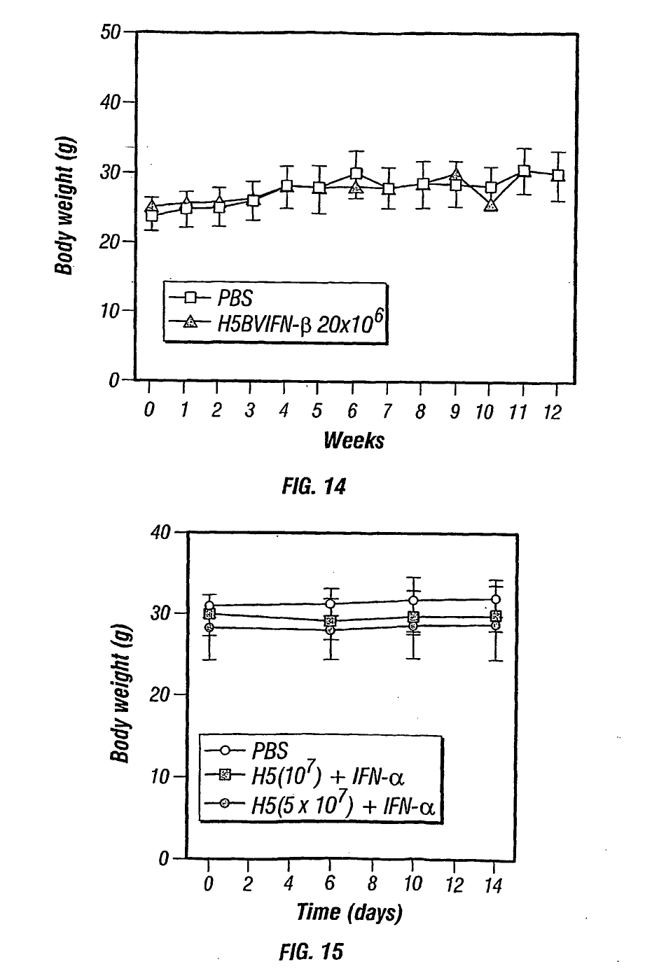

Methods: Two experiments were performed to determine whether subcutaneous administration of H5BVIFN-β produces toxic effects on mice. In the first experiment, normal C3H/HeN mice were randomized into 4 groups (10 mice/group) and injected s.c. with PBS or lyophilized H5BVIFN-β (2×10⁶, 20×10⁶, or 40×10⁶ cells/injection) for 2 times 1 week apart. Body weight of each mouse was measured once for 6 weeks (FIG. 13). After 6 weeks, three mice per group were euthanized and lungs, liver, kidneys, spleen, heart, brain, and a fragment of small intestine were collected for each mouse for histologic study. In the second experiment, potential toxic effects of long-term administration of H5BVIFN-β were determined. C3H mice were randomized into 3 groups (10 mice/group) and injected s.c. with PBS or with lyophilized preparation of 20×10⁶H5BVIFN-β in 100 μl PBS/mouse once a week for 6 weeks or 12 weeks. Body weight of each mouse was measured once a week (FIG. 14). After 6 weeks or 12 weeks, three mice per group were euthanized and lungs, liver, kidneys, spleen, heart, brain, and a fragment of small intestine were collected for each mouse for histologic study.

Conclusions: Two consecutive injections (once a week for 2 weeks) of H5BVIFN-β at doses up to 4×10⁷ H5BVIFN-β, which is 20 times as that used in therapy studies, or 12 consecutive injections (once a week for 12 weeks) of 2×10⁷ H5BVIFN-β did not significantly alter mouse body weight (FIGS. 13 and 14). At the end of the 6th week or the 12th week, mice were sacrificed and several internal organs were sampled for H & E-staining. The treatment H5BVIFN-β did not cause significant changes in the morphology of brain, heart, intestine, kidney, liver, lung, and spleen. These data conclude that administration of H5BVIFN-β at 100 times of the therapeutic doses has no significant toxicity to C3H/HeN mice.

Methods: C3H/HeN female mice at 12 weeks of age were divided into six groups: Groups 1-3 were tumor-bearing mice (5 mice per group), and Groups 4-6 were normal mice (5 mice per group). Tumor-bearing mice were injected with UV-2237m cells s.c. For each mouse, 4 sites were injected. When each tumor reached approximately 1 cm in diameter, mice were injected with materials detailed in the treatment section. Treatment was as follows: Groups 1 and 4 were treated 1 ml of PBS; Groups 2 and 5 were treated with 1 ml of PBS with 10⁷ lyophilized H5 cells plus 2×10⁴ units of murine IFN-α; Groups 3 and 6 were treated with 1 ml of PBS with 5×10⁷ lyophilized H5 cells plus 2×10⁴ units of murine IFN-α.

Conclusions: Tumor-bearing mice: The mice were monitored for 1 week after the intratumoral injection. No toxicity was found and there was no significant change in behavior. Normal mice: After the intraperitoneal injection, the mice were monitored for 2 weeks. No toxicity was found. Body weight was unaltered (see FIG. 15). Thus, injection of the mixture of lyophilized H5 cells and IFN-α, either directly into s.c. tumors (tumor-bearing mice) or peritoneal cavity (normal mice), did not produce any noticeable toxic effects on mice.

All of the compositions and methods disclosed and claimed herein can be made and executed without undue experimentation in light of the present disclosure. While the compositions and methods of this invention may have been described in particular terms, those of skill in the art appreciate that variations of these compositions, and in the steps or in the sequence of steps of the methods described herein, may be practiced without departing from the concept, spirit and scope of the invention. More specifically, it will be apparent that agents which are chemically and/or physiologically related may be substituted for the agents described herein while the same or similar results would be achieved.

### I. REFERENCES

The following references, to the extent that they provide exemplary procedural or other details supplementary to those set forth herein, are specifically incorporated herein by reference.
U.S. Pat. No. 4,430,434
U.S. Pat. No. 4,554,101
U.S. Pat. No. 4,559,302
U.S. Pat. No. 4,578,770
U.S. Pat. No. 4,596,792
U.S. Pat. No. 4,599,230
U.S. Pat. No. 4,599,231
U.S. Pat. No. 4,601,903
U.S. Pat. No. 4,608,251
U.S. Pat. No. 4,631,211
U.S. Pat. No. 4,708,781
U.S. Pat. No. 4,727,028
U.S. Pat. No. 4,745,051
U.S. Pat. No. 4,879,236
U.S. Pat. No. 4,960,704
U.S. Pat. No. 5,077,214
U.S. Pat. No. 5,155,037
U.S. Pat. No. 5,162,222
U.S. Pat. No. 5,169,784
U.S. Pat. No. 5,278,050
U.S. Pat. No. 5,498,540
U.S. Pat. No. 5,759,809
U.S. Pat. No. 5,851,984
U.S. Pat. No. 6,218,371
U.S. Pat. No. 6,339,068
U.S. Pat. No. 6,406,705
Aderem and Ulevitch, Nature, 406:782-787, 2000.
Akira et al., Nat. Immunol., 2:675-680, 2001.
Alt et al., J. Biol. Chem., 253:1357-1320, 1978.
Ashley et al., J. Neuroimmunol., 78:34-46, 1997.
Ayres et al., Virology 202:586-605, 1994.
Ballas et al., J Immunol., 157(5):1840-5, 1996.
Bangham et al., J. Mol. Biol., 13:238, 1965.
Benne et al., J. Clin. Invest., 76:2182, 1995.
Bird, Trends in Genetics, 3:342-347, 1987.
Blissard and Rohrmann, Annu. Rev. Entomol. 35:127-155, 1990.
Blissard and Rohrmann, Virology 170:537-555, 1989.
Brutlag et al., CABIOS, 6:237-245, 1990.
Carson et al., J. Virol., 65:945-951, 1991.
Chalfie et al., Science, 263:802-805, 1994.
Charlton and Volkman, Virology, 197, 245-254, 1993.
Chou and Fasman, Adv. Enzymol. Relat. Areas Mol. Biol., 47:45-148, 1978a.
Chou and Fasman, Ann. Rev. Biochem., 47:251-276, 1978b.
Chou and Fasman, Biochemistry, 13(2):211-222, 1974b.
Chou and Fasman, Biochemistry, 13(2):222-245, 1974a.
Chou and Fasman, Biophys. J., 26:367-384, 1979.
Colberre-Garapin et al., J. Mol. Biol., 150:1-14, 1981.
Cowdery et al., J. Immunol., 156(12):4570-5, 1996.
Davis et al., J. Immunol., 160:870-876, 1998.
Deamer and Uster, LIPOSOMES, M. Ostro ed., 1983.
Fakhrai et al., Proc. Nat'l. Acad. Sci. USA, 93: 2909-2914, 1996.
Fetrow and Bryant, Biotech., 11:479-483, 1993.
Fidler et al., Cancer Metastasis Rev., 18:387-400, 1999.
Fidler, Cancer Chemother. Pharmacol. 43 Suppl:S3-10, 1999.
Galea-Lauri et al., Cancer Gene Ther., 3:202-214, 1996.
Geysen et al., J. Immunol. Methods, 102(2):259-74, 1987.
Geysen et al., Proc Nat'l Acad Sci USA, 81(13):3998-4002, 1984.
Ghosh and Bachhawat, “Targeting of liposomes to hepatocytes,” In: Wu G. Wu C ed., Liver diseases, targeted diagnosis and therapy using specific receptors and ligands, New York: Marel Dekker, pp. 87-104, 1991.
Gregoriadis, Drug Carriers In Biology And Medicine, G. Gregoriadis (ed.), 1979, pp. 287-341.
Grooms et al., J. Surg. Oncol., 9:147-153, 1977.
Guarino and Summers, J. Virol., 61:2091-2099, 1987.
Guarino et al., J. Virol., 60:224-229, 1986.
Halpern et al., Cell Immunol, 167(1):72-8, 1996.
Hooft van Iddekinge et al., Virology, 131:561-565, 1983.
Houghten, Proc. Nat'l. Acad. Sci. USA, 82:5131-5135, 1985.
Jaffee, Ann. NY Acad. Sci., 886:67-72, 1999.
Jameson and Wolf, Comput. Appl. Biosci., 4(1):181-186, 1988.
Jaroszewski and Cohen, Ad. Drug. Del. Rev., 6:235, 1991.
Kadowaki et al., J. Exp. Med., 194:863-869, 2001.
Kaneda et al., Science, 243:375-378, 1989.
Kaplan and Meier, J. Am. Stat. Assoc., 53:457-481, 1958.
Kato et al., J. Biol. Chem., 266:3361-3364, 1991.
Kaufman, Methods Enzymol., 185:537-566, 1990.
Kidd and Emery, Appl. Biochem. Biotechnol., 42:137-159, 1993.
Klinman et al., Proc. Natl. Acad. Sci. USA, 2879-2883, 1996.
Krieg et al., Nature, 374:546-549, 1995.
Krieg et al., Antisense Research and Development, 1:161, 1991.
Kripke, J. Nat'l Cancer Inst., 63:541-548, 1979.
Kuzio et al., Virology, 139:414-418, 1984.
Lewis, Cancer, 61:593-601, 1988.
Loeffler et al., In: Cancer: Principles and Practice of Oncology, Vincent et al. (eds.), 5^{th} Ed., 2523-2606, NY, Lippincott-Raven, 1997.
Lu et al., Cancer Res., 59:5202-5208, 1999.
Lu et al., Int. J. Cancer, 100:480-485, 2002.
Martignoni et al., J. Econ. Entomol., 75:1120-1124, 1982.
Medzhitov, Nature Rev. Immunol., 1:135-145, 2001.
Messina et al., J. Immunol., 147:1759-1764, 1991.
Mitchell, J. Clin. Oncol., 7:1701-1709, 1989.
Murphy and Sturm, J. Exp. Med., 38:183, 1923.
Nicolau et al., Methods Enzymol., 149:157-176, 1987.
Okada et al., Int. J. Cancer, 78:196-201, 1998.
O'Reilly et al., In: Baculovirus Expression Vectors, Freeman and Company, NY, 1992.
Ostrand-Rosenberg et al., Immunol. Rev., 170:101-114, 1999.
Pardoll, Immunol. Today, 14:310-316, 1993.
Peng et al., Int. Immunol., 13: 3-11, 2001.
Posner, Rev. Neurol., 148:477-487, 1992.
Possee, Curr. Opin. Biotechnol., 8:569-572, 1997.
Raz et al., J. Nat'l Cancer Inst., 66:183-189, 1981.
Remington's Pharmaceutical Sciences, 15th Edition, pages 1035-1038, 1570-1580, 1990.
Rosenberg, Immunol. Today, 18:175-182, 1997.
Rosenberg, J. Intern. Med., 250:462-475, 2001.
Sampson et al., Proc. Nat'l Acad. Sci. USA, 93: 10399-10404, 1996.
Santerre et al., Gene, 30:147-156, 1984.
Schackert and Fidler, Int. J. Cancer, 41:589-594, 1988.
Shirai, Jpn. Med. World, 1:14-15, 1921.
Sibille, et al., J. Ex. Med., 172:35-45, 1990.
Summers and Smith, Tex. Agric. Exp. Stn. Bull., No. 1555, 1987.
Sun and Sprent, Curr. Topics Microbiol. Immunol., 247: 107-117, 2000.
Sun et al., J. Exp. Med., 188:2335-2342, 1998.
Szoka and Papahadjopoulos, Proc. Nat'l Acad. Sci. U.S.A. 75:4194-98, 1978.
Talmadge and Fidler, J. Nat'l Cancer Inst., 69:975-980, 1982.
Thiem and Miller, J. Virol., 63:2008-2018, 1989.
Tokanuga et al., JNCI, 72:955, 1994.
Tokanuga et al., Jpn. J. Cancer Res., 79:682-686, 1988.
Visse et al., Cancer Genre Ther., 6:37-44, 1999.
Volkman et al., Virology, 148:288-297, 1986.
Weidner et al., N. Engl. J. Med., 324: 1-8. 1991.
Weinberger et al., Science, 228:740-742, 1985.
Wekerle et al., J. Exp. Biol., 132:43-57, 1987.
Whitford et al., J. Virol., 63:1393-1399, 1989.
Wolf et al., Comput. Appl. Biosci., 4(1):187-191, 1988.
Yamamoto et al., J. Immunol., 12:4072-4076, 1992.
Yi et al., J. Immunol., 156:558-564, 1996.
Zucker et al., Surg. Neurol., 9:177-180, 1978.

1. A method for treating a subject with occult brain metastasis comprising administering to said subject a composition comprising an immunomodulatory polypeptide and a baculovirus-insect cell preparation.

2. The method of claim 1, wherein the composition is injected directly into a tumor or into tumor vasculature not located in the brain.

3. The method of claim 1, wherein said immunomodulatory polypeptide was expressed from a recombinant baculovirus vector in an insect cell.

4. The method of claim 1, wherein the immunomodulatory polypeptide is IFN-α, IFN-β, IFN-γ, IL-1, IL-2, IL-6, IL-7, IL-12, IL-15, IL-16 or GM-CSF.

5. The method of claim 1, wherein the composition further comprises an inflammatory stimulus.

6. The method of claim 5, wherein the inflammatory stimulus is whole bacteria, endotoxin, or unmethylated DNA.

7. The method of claim 1, wherein said composition comprises Spodoptera or Trichoplusia cells.

8. The method of claim 1, further comprising a second administration of said composition.

9. The method of claim 8, further comprising a third administration of said composition.

10. The method of claim 1, wherein said composition comprises between about 10⁵ and about 10⁷ insect cells.

11. The method of claim 10, wherein said composition comprises intact insect cells.

12. The method of claim 10, wherein said composition comprises disrupted insect cells.

13. The method of claim 1, wherein said composition is lyophilized.

14. The method of claim 12, wherein said composition has been freeze/thawed.

15. The method of claim 1, wherein the occult brain metastasis is derived from a primary tumor in said subject's bone, liver, spleen, pancreas, lung, colon, testis, ovary, breast, cervix, prostate, and uterus.

16. The method of claim 1, wherein said composition further comprises a tumor antigen.

17. The method of claim 16, wherein said tumor antigen is MAGE-1, MAGE-3, Melan-A, P198, P1A, gp100, TAG-72, p185^{HER2}, milk mucin core protein, carcinoembryonic antigen (CEA), P91A, p53, p21^{ras}, P210, BTA or tyrosinase.

18. The method of claim 17, wherein said tumor antigen was expressed from a recombinant baculovirus vector in an insect cell.

19. The method of claim 1, wherein said subject is a human subject.

20. The method of claim 1, further comprising a second anti-cancer therapy.

21. The method of claim 20, wherein said second anti-cancer therapy is radiotherapy, chemotherapy, gene therapy or surgery.

22. The method of claim 1, wherein said subject has previously received cancer therapy.

23. A method for preventing the development of occult brain metastasis in a subject comprising administering to said subject a composition comprising an immunomodulatory polypeptide and a baculovirus-insect cell preparation.

24. A method for treating a subject with occult brain metastasis comprising administering to said subject a composition comprising an immunomodulatory polypeptide and an inflammatory stimulus.

25. The method of claim 24, wherein the composition is injected directly into a tumor or into tumor vasculature not located in the brain.

26. The method of claim 24, wherein said immunomodulatory polypeptide was expressed from a recombinant baculovirus vector in an insect cell.

27. The method of claim 24, wherein the immunomodulatory polypeptide is IFN-α, IFN-β, IFN-γ, IL-1, IL-2, IL-6, IL-7, IL-12, IL-15, IL-16 or GM-CSF.

28. The method of claim 24, wherein the inflammatory stimulus is whole bacteria, endotoxin, or unmethylated DNA.

29. The method of claim 24, wherein said composition comprises Spodoptera or Trichoplusia cells.

30. The method of claim 24, further comprising a second administration of said composition.

31. The method of claim 30, further comprising a third administration of said composition.

32. The method of claim 24, wherein said composition is lyophilized.

33. The method of claim 24, wherein said composition has been freeze/thawed.

34. The method of claim 24, wherein the occult brain metastasis is derived from a primary tumor in said subject's bone, liver, spleen, pancreas, lung, colon, testis, ovary, breast, cervix, prostate, and uterus.

35. The method of claim 24, wherein said composition further comprises a tumor antigen.

36. The method of claim 35, wherein said tumor antigen is MAGE-1, MAGE-3, Melan-A, P198, P1A, gp100, TAG-72, p185^{HER2}, milk mucin core protein, carcinoembryonic antigen (CEA), P91A, p53, p21^{ras}, P210, BTA or tyrosinase.

37. The method of claim 36, wherein said tumor antigen was expressed from a recombinant baculovirus vector in an insect cell.

38. The method of claim 24, wherein said subject is a human subject.

39. The method of claim 24, further comprising a second anti-cancer therapy.

40. The method of claim 39, wherein said second anti-cancer therapy is radiotherapy, chemotherapy, gene therapy or surgery.

41. The method of claim 24, wherein said subject has previously received cancer therapy.

42. A method for preventing the development of occult brain metastasis in a subject comprising administering to said subject a composition comprising an immunomodulatory polypeptide and an inflarmmatory stimulus.
